(19) United States
(12) Patent Application Publication
(10) Pub. No.: US 2025/0032551 A1
(43) Pub. Date: Jan. 30, 2025

(54) METHOD FOR PRODUCING NEURAL CREST CELLS SPECIALIZED FOR DIFFERENTIATION INTO MESENCHYMAL LINEAGE

(71) Applicant: Kyoto University, Kyoto-shi, Kyoto (JP)

(72) Inventors: Makoto IKEYA, Kyoto (JP); Daisuke KAMIYA, Kyoto (JP)

(73) Assignee: Kyoto University, Kyoto-shi, Kyoto (JP)

(21) Appl. No.: 18/717,354

(22) PCT Filed: Nov. 25, 2022
(86) PCT No.: PCT/JP2022/043474

§ 371 Date: Jun. 6, 2024

(30) Foreign Application Priority Data
Dec. 6, 2021 (JP) 2021-198151

(51) Int. Cl. A61K 35/28; A61P 21/00; C12N 5/0775

(52) U.S. Cl. CPC A61K 35/28; A61P 21/00; C12N 5/0662; C12N 2500/98; C12N 2501/11; C12N 2501/115; C12N 2506/45

(57) ABSTRACT

The present invention provides a method for producing, from pluripotent stem cells, neural crest cells specialized for differentiation into a mesenchymal lineage, wherein the method includes: 1) a step for culturing pluripotent stem cells under xeno-free and feeder-free conditions in culture broth containing an ALK inhibitor and a GSK-3β inhibitor to obtain neural crest cells; and 2) a step for culturing the neural crest cells under xeno-free and feeder-free conditions in culture broth that includes an ALK inhibitor, EGF, and FGF and is substantially free of GSK-3β inhibitor.

### FIELD

The present invention relates to a method for producing neural crest cells specialized for differentiation into a mesenchymal lineage. More specifically, the present invention relates to a method for producing neural crest cells specialized for differentiation into a mesenchymal lineage, comprising a step in which neural crest cells derived from pluripotent stem cells are cultured under specific conditions.

### BACKGROUND

Mesenchymal stem cells (MSC), also known as mesenchymal stromal cells, are a cell population that can differentiate into osteocytes, chondrocytes and adipocytes in vitro, and the MSCs are found in several tissues including bone marrow, adipose tissue, synovial membrane, dental pulp and umbilical cord blood tissue. Since bone marrow-derived MSCs have long been used for hematopoietic stem cell transplantations, MSCs are considered safe for medical purposes. Primary culture MSCs are currently used for treatment of several clinical complications of graft-versus-host disease, Crohn disease, ischemic cardiomyopathy and cerebral apoplexy. The wide scope of their potential applications demonstrates the importance of MSCs for cell therapy (NPL 1).

MSC quality varies depending on the donor's state of health and the dosage. Moreover, because MSC production in the body decreases with age, typically fewer MSCs can be obtained from elderly persons. It is therefore essential to develop a method for stably supplying high-quality MSCs for cell therapy. Many efforts have been made toward this goal (such as improvements in culture media and optimization of oxygen conditions), but no method has yet been established for stably supplying high-quality MSCs.

In developmental terms, MSCs are known to differentiate via mesodermal cells or neural crest cells (NCC) (see NPL 2, for example). NCCs are pluripotent ectodermal cells generated from the intermediate region between the neuroectoderm and epidermal ectoderm during vertebrate development, and the NCCs are known to differentiate into ectodermal lineage cells such as peripheral neurons and glial cells, mesoderm cells such as osteocytes, chondrocytes and adipocytes derived from MSCs, and endoderm cells such as hepatocytes and pancreatic cells. Based on this knowledge, the present inventors have previously developed a simple and robust method for inducing MSCs from human induced pluripotent stem cells (iPSC), via NCCs (NPL 3). However, because this induction method involves animal-derived components it has been unsuitable for cell therapy.

Incidentally, the present inventors have also previously developed a method for expansion culturing of NCCs while maintaining their properties (PTL 1). Specifically, neural crest cells induced to differentiate from pluripotent stem cells were cultured in medium containing a GSK3β inhibitor at a concentration exhibiting the same effect as that exhibited by CHIR99021 at a concentration exceeding 1 μM, and as a result succeeded in achieving expansion culturing of neural crest cells while maintaining their multipotency. However, neural crest cells specialized for differentiation into a mesenchymal lineage are not disclosed in PTL 1.

### CITATION LIST

### Patent Literature

[PTL 1] International Patent Publication No. WO2019/107485

### Non Patent Literature

[NPL 1] Mendicino M. et al., Cell Stem Cell 14, 141-145 (2014)
[NPL 2] Sheng G., BMC Dev Biol 15, 44 (2015)
[NPL 3] Fukuta M. et al., PLoS One 9, e112291 (2014)

### SUMMARY

### Technical Problem

It is therefore an object of the present invention to provide a method for obtaining mesenchymal stem cells such as bone, cartilage and muscle, suitable for use in regenerative medicine, which do not include heterogenous animal components (i.e. are xeno-free), allowing their use in cell therapy, and to also provide as a preliminary stage, a method for obtaining neural crest cells specialized for differentiation into a mesenchymal lineage from pluripotent stem cells in a xeno-free manner.

### Solution to Problem

The present inventors have researched methods for producing mesenchymal stem cells (MSC) from pluripotent stem cells via neural crest cells (NCC), and by improving the method described in NPL 3, have developed a method for inducing differentiation from pluripotent stem cells to NCCs and a method for inducing differentiation of MSCs from the NCCs, under xeno-free conditions. However, it was found that when maintenance culturing is carried out under xeno-free conditions, the NCCs induced to differentiate by this method fluctuate in their differentiation potency with subsequent subculturing. NCCs are cells capable of differentiating into a variety of cell types such as bone, cartilage, muscle, sensory nerves, glial cells, melanocytes and adipocytes, but it was found that when NCCs are maintenance cultured in xeno-free medium containing an ALK inhibitor, EGF and FGF, the NCCs lose differentiation potency to nervous system cells and melanocytes after a certain number of subcultures. In other words, the present inventors have succeeded in creating novel neural crest cells without differentiation potency into nervous system cells or melanocytes.

More surprisingly, it was found that inducing differentiation of the novel neural crest cells into MSCs can yield MSCs with a high survival rate and high differentiation efficiency, and that transplanting the MSCs at the defective sites of mice with partial loss of cranial bone or muscle leads to efficient repair of the cranial bone and muscle. The present inventors have completed this invention as a result of much research conducted on the basis of these findings.

Specifically, the present invention is as follows.

[1]

A method for producing neural crest cells specialized for differentiation into a mesenchymal lineage from pluripotent stem cells, comprising:
1) a step of culturing pluripotent stem cells in culture medium containing an ALK inhibitor and a GSK-3β inhibitor under xeno-free and feeder-free conditions to obtain neural crest cells, and
2) a step of culturing the neural crest cells in culture medium containing an ALK inhibitor, EGF and FGF but containing substantially no GSK-3β inhibitor, under xeno-free and feeder-free conditions.
   [2]

The method according to [1] above, wherein the culturing period in step 2) is 7 to 45 days.

[3]

The method according to [1] or [2], wherein at least one ALK inhibitor used in step 2) is SB431542.

[4-1]

The method according to any one of [1] to [3] above, wherein the culturing in step 1) and/or step 2) is adhesion culturing.

[4-2]

The method according to [4-1] above, wherein the adhesion culturing is carried out in a culture vessel coated with fibronectin.

[5]

The method according to any one of [1] to [4-2], wherein the pluripotent stem cells are induced pluripotent stem cells or embryonic stem cells.

[6]

The method according to any one of [1] to [5] above, wherein the pluripotent stem cells are derived from a human.

[7]

Neural crest cells obtained by the method according to any one of [1] to [6] above.

[8]

Neural crest cells having the following features (A) to (C):
(A) the cells are derived from pluripotent stem cells,
(B) the cells express one or more genes selected from among TWIST, DLX1 and CDH11,
(C) the cells do not express PAX3 and/or SOX10.
   [9]

Neural crest cells according to [8] above, which also have the following features (D) and/or (E):
(D) the cells do not have differentiation potency to nervous system cells,
(E) the cells do not have differentiation potency to melanocytes.
   [10]

A method for producing mesenchymal stem cells, comprising a step of culturing neural crest cells according to any one of [7] to [9] above in mesenchymal stem cell differentiation-inducing medium.

[11]

Mesenchymal stem cells obtained by the method according to [10] above.

[12]

A method for producing mesenchymal cells, comprising a step of culturing mesenchymal stem cells according to [11] above in mesenchymal cell differentiation-inducing medium.

[13-1]

Mesenchymal cells obtained by the method according to [12] above.

[13-2]

Mesenchymal cells according to [13-1] above, wherein the mesenchymal cells are osteocytes, chondrocytes or adipocytes.

[14]

An agent for cell transplantation therapy comprising mesenchymal stem cells according to [11] above or mesenchymal cells according to [13-1] or [13-2] above.

[15]

The agent for cell transplantation therapy according to [14] above, which is for treatment of tissue damage or disease.

[16]

A method for treating tissue damage or disease, comprising administrating or transplanting an effective amount of mesenchymal stem cells according to [11] above or mesenchymal cells according to [13-1] or [13-2] above.

[17]

Mesenchymal stem cells according to [11] above or mesenchymal cells according to [13-1] or [13-2] above, which are for use in treatment of tissue damage or disease.

[18]

The use of mesenchymal stem cells according to [11] above or mesenchymal cells according to [13-1] or [13-2] above for production of a therapeutic agent for tissue damage or disease.

### Advantageous Effects of Invention

According to the present invention it is possible to produce neural crest cells specialized for differentiation into a mesenchymal lineage under xeno-free conditions, the neural crest cells being useful as starting cells for mesenchymal stem cells that are important for cell transplanting therapy. Since proliferation culture and cryopreservation are possible at the neural crest cell stage, it is easy to ensure the necessary cell count for cell transplantation therapy, while quality inspection is also possible at this stage. It is also possible to produce mesenchymal stem cells from the neural crest cells under xeno-free conditions. With a route via neural crest cells it is possible to lower the risk of contamination of undifferentiated iPS cells into the mesenchymal stem cells. In addition, inducing differentiation of mesenchymal stem cells from neural crest cells of the same lot allows production of mesenchymal stem cells of consistent quality, thus making the mesenchymal stem cells particularly suitable for application to cell transplantation therapy. For example, mesenchymal stem cells obtained according to the present invention can be suitably used for treatment of diseases associated with damage to bone, cartilage or muscle (cell transplantation therapy).

### BRIEF DESCRIPTION OF DRAWINGS

FIG. 1 Efficient induction of iNCCs from iPSCs under xeno-free conditions. (A) Schematic representation of a neural crest induction protocol using AK03N and Basic03 xeno-free medium. (B) Morphology of colonies during the induction. Phase contrast images obtained on days 0, 4, and 10. In the far-right panel, SOX10 (red color) expression was merged at day 10. Scale bars, 200 μm. (C) Numbers of cells during neural crest induction. Data are the mean±SD, n=3. (D) Percentage of CD271-positive cells during neural crest induction, as analyzed by FCM. Data are the mean±SD, n=3. (E) The expression of marker genes in sorted CD271^{high} (CD271H) and CD271^{low} (CD271L) cells. The mRNA expression of each gene was analyzed by RT-qPCR in 1231A3 iPSCs and CD271H and CD271L cells and is shown relative to the expression level (used as 1.0) in 1231A3 iPSCs. Data are the mean±SD, n=3. **P<0.01. n.s. not significant. Neuron (F), glial cell (G), and melanocyte (H) differentiation from CD271H-sorted cells. (F) Cells were stained with an anti-TUBB3 antibody (green) and anti-peripherin antibody (red). (G) Cells were stained with an anti-GFAP antibody (green) and anti-peripherin antibody (red). (H) Cells were stained with an anti-MITF antibody (red). Scale bars, 50 μm (F, G, H).

FIG. 2 Successful induction of iNCCs from iPSCs. (A) Immunofluorescence images of the NCC induction at day 10. Cells were stained with anti-SOX10 antibody (left and middle panels, green), anti-CD271 antibody (left panel, red), and anti-TFAP2A antibody (right and middle panels, red). Nuclei were stained with DAPI (blue). Scale bar, 50 μm. (B) The fraction of cells with high CD271 expression (CD271H) on day 10 of NCC induction (dark gray) of 1231A3 iPSCs. The isotype control is gray. (C) A dot plot analysis of the NCC induction at day 10. The x-axis indicates CD271 expression, and the y-axis indicates SSEA4 expression. (D) Fraction of the CD271H population on day 10 of the NCC induction of various iPSC lines (1231A3, 1381A5, 1381B5, 1382D2, 1383D10). Data are the mean±SD, n=3. (E) The fraction of CD271H cells after sorting.

FIG. 3 Global gene expression profile reveals the stepwise differentiation of iNCCs from iPSCs through ectodermal lineage. (A) A heatmap illustrating the expression of pluripotent stem cell (PSC), neuroectoderm (NE), neural plate border (NPB), neural crest cell (NCC), and ectoderm (ECT) genes by 1231A3 iPSCs, induced NCCs on days 2-10 (D2, D4, D6, D8, D10), and the high (H) and low (L) fractions of CD271-positive cells. (B) A heatmap illustrating the expression of mesoderm (MES) and endoderm (END) genes for 1231A3 iPSCs, induced NCCs on days 2-10 (D2, D4, D6, D8, D10), and the high (H) and low (L) fractions of CD271-positive cells. (C) A heatmap illustrating the expression of regional markers, forebrain (FB), midbrain (MB), midbrain hindbrain border (MHB), hindbrain (HB), and spinal cord (SC) genes for 1231A3 iPSCs, induced NCCs on days 2-10 (D2, D4, D6, D8, D10), and high (H) and low (L) fractions of CD271-positive cells. (D) A PCA analysis of NCC induction from 1231A3 iPSCs. PC1: principal component 1, PC2: principal component 2.

FIG. 4 Xeno-free conditions to expand iNCCs that have lost neural differentiation potency. (A) Schematic representation of the neural crest expansion culture protocol. (B) Phase contrast images of PNs 0, 2, 4, and 7. Scale bars, 100 μm. (C) Number of cells during the expansion culture. Data are the mean±SD, n=3. (D) The expression of marker genes during the expansion culture. The mRNA expression of each gene was analyzed by RT-qPCR during the expansion culture and is shown relative to the expression level (used as 1.0) in 1231A3h iPSCs. Data are the mean±SD, n=3. (E) Immunostaining of SOX10 (purple) TWIST (green) and DLX1 (red) during the expansion culture. Scale bar, 100 μm. (F) Peripheral neuron differentiation from PN1 (upper panel) or PN4 (lower panel) of the expansion culture. Cells were stained with anti-TUBB3 antibody (green) and anti-Peripherin antibody (red). Nuclei were stained with DAPI (blue). Scale bar, 200 μm.

FIG. 5 Xeno-free induction of iMSCs from expanded iNCCs. (A) Schematic representation of the mesenchymal stromal cell (MSC) induction protocol using PRIME-XV MSC XSFM xeno-free medium. (B) Phase contrast images of PNs 1, 2, and 4. Scale bar, 100 μm. (C) Number of cells during the MSC induction. Data are the mean±SD, n=3. (D) The expression of hMSC-related surface markers in XF-iMSCs (dark gray) and isotype control (gray) at passage number 4 (PN4). (E, F, G) Differentiation properties of XF-iMSCs. The induction of chondrogenic, osteogenic, and adipogenic lineages was evaluated by alcian blue staining (E), alizarin red staining (F), and oil red O staining (G), respectively. Scale bar, 50 μm.

FIG. 6 Xf-iMSCs have similar characteristics to adult-derived MSCs. (A) Phase contrast images of XF-iMSCs (PN4), human adipocyte-derived mesenchymal stromal cells (hAC-MSCs), human bone marrow-derived mesenchymal stromal cells (hBM-MSCs), and human umbilical cord-derived MSCs (hUC-MSCs). Scale bar, 200 μm. (B) Hierarchical clustering analysis of XF-iMSCs, hAC-MSCs, hBM-MSCs, and hUC-MSCs. n=3. (C) Heatmap illustrating the expression of pluripotent stem cell (PSC), neural crest cell (NCC), and mesenchymal stromal cell (MSC) genes for 1231A3 iPSCs, induced NCCs on day 10 (NCC), XFiMSCs, hAC-MSCs, hBM-MSCs, and hUC-MSCs. n=2 (iPSCs, NCCs), n=3 (XF-iMSCs, hAC-MSCs, hBM-MSCs, hUC-MSCs). (D) Heatmap illustrating the expression of neural progenitor, neuronal lineage, and glial lineage genes for NCC induction on day 10 (NCCs), XF-iMSCs, hACMSCs, hBM-MSCs, and hUC-MSCs. N=2 (NCC), N=3 (XF-iMSCs, hAC-MSCs, hBM-MSCs, hUC-MSCs).

FIG. 7 Enhanced skull bone regeneration by transplanted osteogenic clump-XF-iMSCs. (A) Schematic representation of the transplantation of XF-iMSCs into mouse skulls. (B) Shapes (upper panel) and sectional images of XF-C-iMSCs stained by HE (lower panel). Scale bars, 5 mm (upper left and right), 100 μm (lower left), and 10 μm (lower right). (C) The expression marker genes in GM cultured clumps (white) and OIM cultured clumps (black). The mRNA expression of each gene was analyzed by RT-qPCR in GM cultured clumps and OIM cultured clumps and is shown relative to the expression level (used as 1.0) in GM cultured clumps. Data are the mean±SD, n=4. **P<0.01. (D) Sectional images of GM and OIM cultured clumps. The clumps were stained by HE (upper panel) or alizarin red (lower panel). Scale bars, 200 μm. (E) CT-scanning images of transplanted mouse skulls. Trepanned areas are indicated by the red circles. (F) Relative bone volume after no graft (white), XF-C-BMMSC transplantation (gray), and XF-C-iMSC transplantation (black). Data are the mean±SD, n=6. **P<0.01. n.s. not significant. (G) Lateral section images of the transplanted skull. Sections were stained by HE (left column), Azan (middle column) or anti-human vimentin (right column). Nuclei were stained with DAPI (blue). Black boxes in the left column show the same areas as those in the middle column in the serial sections. The right column shows high magnification images of the white box in the middle column. Scale bars, 500 μm (left column), 100 μm (middle column), and 10 μm (right column).

FIG. 8 Enhanced skull bone regeneration by transplanted clump (XF-iMSCs). (A) CT-scanning images of the skulls of transplanted mice. Trepanned areas are indicated by the red circles. (B) Relative bone volumes of No graft, XF-C-BMMSCs, and XF-C-iMSCs. Data are mean±SD,n=5. **P<0.01. (C) Lateral section images of the transplanted skulls. Sections were stained by HE or Azan (right bottom). Black boxes in (C) have the same areas as those of the serial sections in (D). Scale bars, 500 μm. (D) Lateral section fluorescence images of transplanted skulls. Sections were stained by anti-human vimentin (green). Nuclei were stained with DAPI (blue). The right column shows high magnification images of the white box in the left column. Scale bar, 100 μm (left column) and 10 μm (right column), respectively.

FIG. 9 Early reactivation of myogenic markers during skeletal muscle regeneration after XF-iMSCs transplantation. (A) Schematic representation of the transplantation of XF-iMSCs into injured skeletal muscle [Tibialis anterior (TA) muscle]. (B) Sectional images of injured TA muscle at 3 days, 2 weeks, and 5 weeks after medium (left column), HDF (middle column), and XF-iMSC (right column) transplantation. Intact: uninjured NSG mouse. Sections were stained by HE. Scale bars, 100 μm. (C) Cross-sectional area per cell in the injured field 2 or 5 weeks after transplantation. Data are the mean±SD, n=3. *P<0.05. n.s. not significant. (D) Sectional images of the injured TA muscle 2 weeks after transplantation. Sections were stained with anti-laminin-α2 antibody (white), anti-MYH4 antibody (red), and anti-human LaminA/C antibody (green). Nuclei were stained with DAPI (blue). Scale bars, 100 μm. (E) Total area of MYH4-positive fibers 2 weeks after transplantation. Data are the mean±SD, n=6. **P<0.01. n.s. not significant. (F) Sectional images of injured TA muscle at 3 days, 1 week, and 2 weeks after transplantation. Sections were stained with anti-MYH3 antibody (red) and anti-human LaminA/C antibody (green). Nuclei were stained with DAPI (blue). (G) Numbers of MYH3-positive cells at 3 days, 1 week, and 2 weeks after transplantation. Data are the mean±SD n=3 (3 days), n=3 (1 week), n=6 (2 weeks). *P<0.05; **P<0.01; n.s. not significant.

FIG. 10 Characteristics of CD271H and CD271L sorted cells. Immunofluorescence images of (A) CD271H sorted cells and (B) CD271L sorted cells at day 10 of the NCC induction. Left panel, cells were stained with an antiTUBB3 antibody (green) and anti-peripherin antibody (red); middle panel, cells were stained with an anti-GFAP antibody (green) and anti-peripherin antibody (red); right panel, cells were stained with an anti-MITF antibody (red). Nuclei were stained with DAPI (blue). Scale bars, 50 μm.

FIG. 11 Gene expression pattern and differentiation ability in NCC expansion culture. (A) A heatmap illustrating the expression of pluripotent stem cell (PSC), premigratory NCC, post-migratory NCC, Pan-NCC and epithelial mesenchymal transition (EMT) marker genes for 1231A3 iPSCs, CD271H population on NCC induction at day 10, and NCC expansion culture passage number (PN) 0, 2, 4, and 7. All samples were n=3. (B) Immunofluorescence images of neural (left) and melanocyte (right)-inducing cells from NCC expansion culture PN1 (upper panel) and PN4 (lower panel). Left panel, cells were stained with an anti-GFAP antibody (green) and anti-peripherin antibody (red); right panel, cells were stained with an anti-MITF antibody (red). Nuclei were stained with DAPI (blue). Scale bars, 50 μm. All sequence data were available in GEO (GSE206128).

FIG. 12 MSC induction and characterization of different NCC expansion culture passage numbers. (A) Phase contrast images of XF-iMSC passage number (PN) 4 from NCC expansion culture PN2 (left), PN4 (middle) and PN7 (right). Scale bars, 200 μm. (B) PCA analysis of the MSC induction from different NCC expansion culture. PC1: principal component 1, PC2: principal component 2. (C) Heatmap illustrating the expression of pluripotent stem cell (PSC), premigratory NCC, post-migratory NCC, Pan-NCC and MSC marker genes for MSCs from NCC expansion culture PN2, 4, 7. All samples were n=4. (D) Phase contrast images of XF-iMSC PN0 from NCC expansion culture PN2 (left) or PN4 (right). Scale bars, 100 μm. All sequence data were available in GEO (GSE206128).

FIG. 13 Long-term culture of NCC. (A) Schematic representation of the NCC expansion culture protocol. (B) NCC expansion culture was carried out for 45 days, after which the expression of hMSC-related surface markers was confirmed with isotype control (top panel) and XF-iMSCs (bottom panel) at passage 4 (PN4).

FIG. 14 Gene expression patterns in XF-iMSCs and human adult derived MSCs. A heatmap illustrating the expression of bone regeneration, muscle regeneration, immunomodulator, secreted growth factors and extra cellular matrix genes for XF-iMSCs, hAC-MSCs, hBM-MSCs and hUC-MSCs. All samples were n=3. All sequence data were available in GEO (GSE206172).

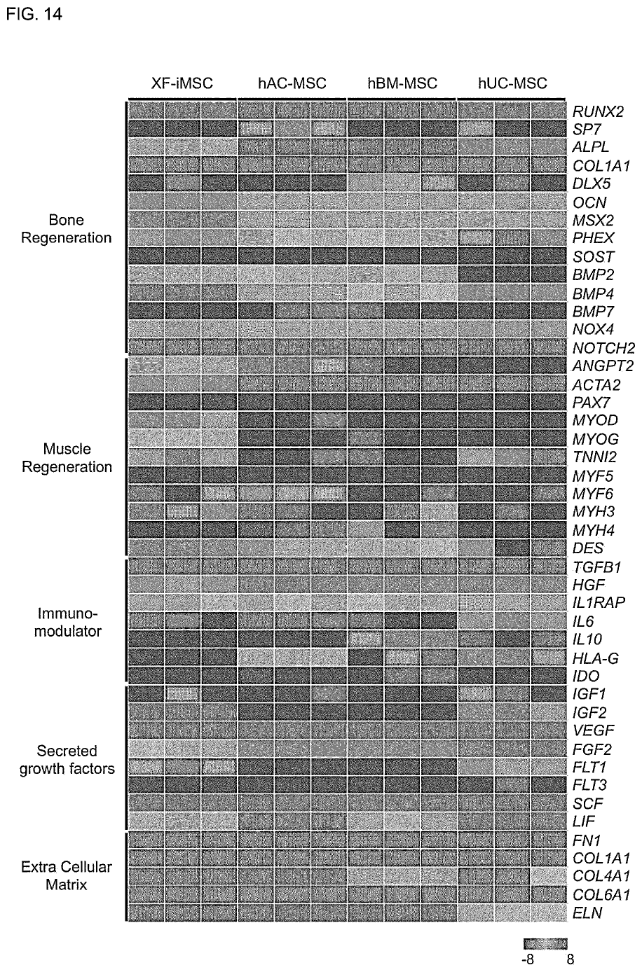

FIG. 15 Skull bone regeneration with transplanted clump-hBMMSCs or clump-XF-iMSCs generated from osteocyte induction medium (OIM). Individual CT scan images of the skull bones of mice transplanted with control (top panel), hBM-C-MSC (middle panel), or XF-C-MSC. All samples were n=6.

FIG. 16 Muscle regeneration by transplanted XF-iMSCs. (A) Sectional images of injured TA muscle 5 weeks after transplantation. Sections were stained with laminin (white), MYH4 (red) and MYH4 (red)/human lamin A/C (green) antibodies. Nuclei were stained with DAPI (blue). Scale bars, 100 μm. (B) Sectional images of injured TA muscle 5 weeks after transplantation. Sections were stained with laminin (white), MYH3 (red)/human lamin A/C (green) antibodies. Nuclei were stained with DAPI (blue). Scale bars, 100 μm.

FIG. 17 Most MYH3-positive cells are found around transplanted XF-iMSCs. Sectional images of injured TA muscle 3 days after XF-iMSCs transplantation. Sections were stained with human lamin A/C (green), MYH3 (red) antibodies. Nuclei were stained with DAPI (blue). White dotted lines indicate the transplanted area. Scale bars, 500 μm.

FIG. 18 Injection of conditioned medium from XF-iMSCs into crushed skeletal muscle. (A) Sectional images of injured TA muscle three days after intact medium (left column) or XF-iMSC-conditioned medium (right column) injection. Sections were stained with antiMYH3 antibody (red). Nuclei were stained with DAPI (blue). Scale bars, 100 μm. (B) Numbers of MYH3-positive cells three days after medium injection. Data are mean±SD n=3. n.s.: not significant.

### DESCRIPTION OF EMBODIMENTS

1. Method for Producing Neural Crest Cells Specialized for Differentiation into a Mesenchymal Lineage

The present invention provides a method for producing neural crest cells specialized for differentiation to a mesenchymal lineage (also referred to as “mesenchymal cell population”) from pluripotent stem cells. Specifically, such method comprises:
1) a step of culturing pluripotent stem cells in culture medium containing an ALK inhibitor and a GSK-3β inhibitor under xeno-free and feeder-free conditions to obtain neural crest cells, and
2) a step of culturing the neural crest cells in culture medium containing an ALK inhibitor, EGF and FGF but containing substantially no GSK-3β inhibitor, under xeno-free and feeder-free conditions (hereunder also referred to as “production method of the present invention”).

The term “pluripotent stem cells” refers to stem cells that can differentiate into tissue or cells having various different forms or functions in the body, and that can also differentiate into cells of any lineages of the three germ layers (endoderm, mesoderm and ectoderm). Examples of pluripotent stem cells to be used for the present invention include induced pluripotent stem cells (iPS cells), embryonic stem cells (ES cells), embryonic stem cells from cloned embryos obtained by nuclear transfer (nuclear transfer Embryonic stem cells; ntES cells), multipotent germline stem cells (mGS cells) and embryonic germ cells (EG cells), of which iPS cells (and especially human iPS cells) are preferred. When the pluripotent stem cells are ES cells or arbitrary cells derived from human embryos, the cells may be obtained by destruction of the embryo or they may be obtained without destruction of the embryo, but preferably they are cells obtained without destruction of the embryo.

ES cells are stem cells having pluripotency and auto-replicating proliferation potency, established from the inner cell mass of an early embryo (such as the blastocyst) of a mammal such as a human or mouse. ES cells were discovered in mice in 1981 (M. J. Evans and M. H. Kaufman (1981), Nature 292:154-156), and ES cell lines were later established in primates including humans and monkeys (J. A. Thomson et al. (1998), Science 282:1145-1147; J. A. Thomson et al. (1995), Proc. Natl. Acad. Sci. USA, 92:7844-7848; J. A. Thomson et al. (1996), Biol. Reprod., 55:254-259; J. A. Thomson and V. S. Marshall (1998), Curr. Top. Dev. Biol., 38:133-165). ES cells can be established by extracting the inner cell mass from the blastocyst of a fertilized egg of a target animal and culturing the inner cell mass on a fibroblast feeder. Alternatively, ES cells can be established using a single embryo blastomere alone during the cleavage stage before the blastocyst stage (Chung Y. et al. (2008), Cell Stem Cell 2: 113-117), or they can be established using a developmentally arrested embryo (Zhang X. et al. (2006), Stem Cells 24: 2669-2676.).

ntES cells are ES cells derived from a clone embryo prepared by nuclear transfer, and they have approximately the same properties as fertilized egg-derived ES cells (Wakayama T. et al. (2001), Science, 292:740-743; S. Wakayama et al. (2005), Biol. Reprod., 72:932-936; Byrne J. et al. (2007), Nature, 450:497-502). In other words, ntES (nuclear transfer ES) cells are ES cells established from the inner cell mass of a cloned embryo-derived blastocyst obtained by exchanging an unfertilized egg nucleus with a somatic cell nucleus. Combinations of nuclear transfer (Cibelli J. B. et al. (1998), Nature Biotechnol., 16:642-646) and ES cell preparation techniques (described above) are used to prepare ntES cells (Wakayama, S. (2008), Jikken Igaku, Vol. 26, No. 5 (special edition), pp. 47-52). For nuclear transfer, a somatic cell nucleus may be implanted into a mammalian enucleated unfertilized egg and cultured for several hours for reprogramming.

The ES cell line used for the present invention may be mouse ES cells, and for example, the different mouse ES cell lines established by the inGenious Targeting Laboratory or Riken (Riken Research Institute) may be used, or for human ES cell lines, the different human ES cell lines established by the University of Wisconsin, NIH, Riken, Kyoto University, the National Center for Child Health and Development or Cellartis, for example, may be used. Specific examples of human ES cell lines include CHB-1 to CHB-12 lines, RUES1 line, RUES2 line and HUES1 to HUES28 lines distributed by ESI Bio Co., H1 and H9 lines distributed by WiCell Research, KhES-1, KhES-2, KhES-3, KhES-4, KhES-5, SSES1, SSES2 and SSES3 distributed by Riken, and so on.

iPS cells are cells obtained by reprogramming of mammalian somatic cells or undifferentiated stem cells by transfer of specific factors (nuclear reprogramming factors). A large number of iPS cells currently exist, among which there may be used iPSCs established by transfer of the 4 factors Oct3/4·Sox2·Klf4·c-Myc into mouse fibroblasts by Yamanaka (Takahashi K, Yamanaka S., Cell, (2006) 126: 663-676), iPSCs derived from human cells established by transfer of the same 4 factors into human fibroblasts (Takahashi K, Yamanaka S., et al. Cell, (2007) 131:861-872.), Nanog-iPSCs established by selecting of Nanog expression markers after transfer of the 4 factors (Okita, K., Ichisaka, T. and Yamanaka, S. (2007). Nature 448, 313-317.), iPSCs produced by methods without c-Myc (Nakagawa M, Yamanaka S., et al. Nature Biotechnology, (2008) 26, 101-106), and iPSCs established by transfer of 6 factors by a virus-free method (Okita K et al. Nat. Methods 2011 May; 8(5):409-12, Okita K et al. Stem Cells. 31(3):458-66.). The induced pluripotent stem cells established by transfer of the 4 factors OCT3/4·SOX2·NANOG·LIN28, created by Thomson et al. (Yu J., Thomson J A. et al., Science (2007) 318: 1917-1920), the induced pluripotent stem cells created by Daley et al. (Park I H, Daley G Q. et al., Nature (2007) 451:141-146) and the induced pluripotent stem cells created by Sakurata et al. (Japanese Unexamined Patent Publication No. 2008-307007) may also be used.

In addition, any induced pluripotent stem cells publicly known in the field as described in published journal (for example, Shi Y., Ding S., et al., Cell Stem Cell, (2008) Vol. 3, Issue 5, 568-574; Kim J B., Scholer H R., et al., Nature, (2008) 454, 646-650; Huangfu D., Melton, D A., et al., Nature Biotechnology, (2008) 26, No 7, 795-797), or patents (for example, Japanese Unexamined Patent Publication No. 2008-307007, Japanese Unexamined Patent Publication No. 2008-283972, US2008-2336610, US2009-047263, WO2007-069666, WO2008-118220, WO2008-124133, WO2008-151058, WO2009-006930, WO2009-006997 and WO2009-007852), may also be used.

Induced pluripotent stem cell lines that are iPSC lines established by NIH, Riken or Kyoto University, for example, may be used as well. Examples include human iPSC lines such as HiPS-RIKEN-1A, HiPS-RIKEN-2A, HiPS-RIKEN-12A and Nips-B2 by Riken, and 253G1, 253G4, 1201C1, 1205D1, 1210B2, 1383D2, 1383D6, 201B7, 409B2, 454E2, 606A1, 610B1, 648A1, 1231A3 and FfI-01s04 by Kyoto University, with 1231A3 being preferred.

mGS cells are pluripotent stem cells derived from the testes, and they serve as a source for spermatogenesis. Similar to ES cells, these cells can also be induced to differentiate to cells of various cell series, and have properties that allow creation of a chimeric mouse when they are grafted into a mouse blastocyst, for example (Kanatsu-Shinohara M. et al. (2003) Biol. Reprod., 69:612-616; Shinohara K. et al. (2004), Cell, 119:1001-1012). The mGS cells are also capable of auto-replication in culture medium containing glial cell line-derived neurotrophic factor (GDNF), and their repeated subculturing under culturing conditions similar to those of ES cells allows germline stem cells to be obtained (Takebayashi, M. et al. (2008), Jikken Igaku, Vol. 26, No. 5 (Special Edition) pp. 41-46, Yodosha (Tokyo, Japan).

EG cells are cells with pluripotency similar to ES cells, being established from embryonic primordial germ cells. The EG cells can be established by culturing primordial germ cells in the presence of substances such as LIF, bFGF and stem cell factors (Matsui Y. et al. (1992), Cell, 70:841-847; J. L. Resnick et al. (1992), Nature, 359:550-551).

The source species of the pluripotent stem cells is not particularly restricted, and for example, the cells may be from a rodent such as a rat, mouse, hamster or guinea pig a lagomorph such as a rabbit, an ungulate such as a pig, cow, goat or sheep, a dog, a feline such as a cat, or a primate such as a human, monkey, rhesus monkey, marmoset, orangutan or chimpanzee. The preferred source species is human.

Embryologically speaking, neural crest cells develop from the region between the neuroectoderm and epidermal ectoderm during formation of the neural tube from the neural plate during initial development, having multipotency and the ability to self-propagate and differentiate into a variety of cell types such as neurons, glial cells, mesenchymal stem cells, osteocytes, chondrocytes and melanocytes. Throughout the present specification, the term “neural crest cells” refers to cells expressing TFAP2A, and the cells also express one or more genes selected from among SOX10, PAX3, NGFR, CDH6, TWIST, DLX1 and CDH11.

The term “cells” as used herein includes “cell populations”, unless otherwise specified. A cell population may be composed of a single type of cell or two or more different types of cells.

Also as used herein, “ALK inhibitor” means a substance having inhibiting activity against receptors belonging to the ALK (activin receptor-like kinase) family. ALK, also known as type I TGFβ receptor, controls cell proliferation, cell differentiation and cell death via signal transduction which is primarily Smad (R-Smad) activation. Type II TGFβ receptors form dimers, the dimers associating with two type I TGF beta receptor molecules to form a heterotetramer. By formation of a heterotetramer, the type II TGFβ receptor phosphorylates type I TGF beta receptor, this phosphorylation inducing kinase activity of the type I TGF beta receptor and downstream phosphorylation of Smad.

The “ALK inhibitor” used for the present invention is not particularly restricted so long as it can inhibit the aforementioned signal transduction at any stage, and for example, it may be a substance that inhibits binding between TGFβ and its receptor, a substance that inhibits phosphorylation of type I TGF beta receptor by type II TGFβ receptor, or a substance that inhibits phosphorylation of Smad (e.g.: Smad2, Smad3, etc.) by phosphorylated type I TGF beta receptor. In humans, ALK-1, ALK-2, ALK-3, ALK-4, ALK-5, ALK-6 and ALK-7 are known ALKs, but the ALK inhibitor used for the present invention is preferably an inhibitor for ALK-5 (also referred to hereunder as “TGFβ inhibitor”), in particular.

Examples of ALK inhibitors to be used in step 1) of the production method of the present invention include SB431542 (4-(5-benzole[1,3]dioxol-5-yl-4-pyridin-2-yl-1H-imidazol-2-yl)-benzamide, 4-[4-(1,3-benzodioxol-5-yl)-5-(2-pyridinyl)-1H-imidazol-2-yl]-benzamide, 4-[4-(3,4-methylenedioxyphenyl)-5-(2-pyridyl)-1H-imidazol-2-yl]-benzamide), A83-01 (3-(6-methylpyridin-2-yl)-1-phenylthiocarbamoyl-4-quinolin-4-ylpyrazole), LDN193189 (4-[6-[4-(1-piperazinyl)phenyl]pyrazolo[1,5-a]pyrimidin-3-yl]-quinoline), GW788388 (4-[4-[3-(pyridin-2-yl)-1H-pyrazol-4-yl]-pyridin-2-yl]-N-(tetrahydro-2H-pyran-4-yl)benzamide), SM16 (4-[4-(1,3-benzodioxol-5-yl)-5-(6-methyl-2-pyridinyl)-1H-imidazol-2-yl]-bicyclo[2.2.2]octane-1-carboxamide), IN-1130 (3-[[5-(6-methyl-2-pyridinyl)-4-(6-quinoxalinyl)-1H-imidazol-2-yl]methyl]-benzamide), GW6604 (2-phenyl-4-[3-(pyridin-2-yl)-1H-pyrazol-4-yl]pyridine) and SB505124 (2-(5-benzo[1,3]dioxol-5-yl-2-tert-butyl-3H-imidazol-4-yl)-6-methylpyridine). These may also be used in combinations of two or more.

In step 1) of the production method of the present invention, the concentration of the ALK inhibitor in the medium may be appropriately adjusted depending on the type of ALK inhibitor added, but it will typically be 1 to 50 μM, preferably 2 to 40 μM and more preferably 5 to 20 μM. When SB431542 (4-[4-(1,3-benzodioxol-5-yl)-5-(2-pyridinyl)-1H-imidazol-2-yl]-benzamide) is used, its concentration in the medium will typically be 1 to 40 μM, preferably 5 to 20 μM and more preferably 10 μM.

As used herein, “GSK3β inhibitor” means a substance with inhibiting activity against GSK3β (Glycogen Synthase Kinase 3β). GSK3 (Glycogen Synthase Kinase 3) is a type of serine/threonine protein kinase which is involved in numerous signal pathways associated with glycogen production and apoptosis, stem cell maintenance and so on. GSK3 has the two isoforms α and β. The “GSK3β inhibitor” used for the present invention is not particularly restricted so long as it has GSK3β inhibiting activity, and it may also be a substance having both GSK3β inhibiting activity and GSK3α inhibiting activity.

Examples of GSK3β inhibitors to be used in step 1) of the production method of the present invention include CHIR98014 (2-[[2-[(5-nitro-6-aminopyridin-2-yl)amino]ethyl]amino]-4-(2,4-dichlorophenyl)-5-(1H-imidazol-1-yl)pyrimidine), CHIR99021 (6-[[2-[[4-(2,4-dichlorophenyl)-5-(4-methyl-1H-imidazol-2-yl)-2-pyrimidinyl]amino]ethyl]amino]nicotinonitrile), CP21R7 (3-(3-amino-phenyl)-4-(1-methyl-1H-indol-3-yl)-pyrrole-2,5-dione), LY2090314 (3-[9-fluoro-1,2,3,4-tetrahydro-2-(1-piperidinylcarbonyl)pyrrolo[3,2,1-jk][1,4]benzodiazepin-7-yl]-4-imidazo[1,2-a]pyridin-3-yl-1 h-pyrrole-2,5-dione), TDZD-8 (4-benzyl-2-methyl-1,2,4-thiadiazolysine-3,5-dione), SB216763 (3-(2,4-dichlorophenyl)-4-(1-methyl-1H-indol-3-yl)-1H-pyrrole-2,5-dione), TWS-119 (3-[6-(3-aminophenyl)-7H-pyrrolo[2,3-d]pyrimidin-4-yloxy]phenol), Kenpaullone, 1-Azakenpaullone, (Azakenpaullone), SB415286 (3-[(3-chloro-4-hydroxyphenyl)amino]-4-(2-nitrophenyl)-1H-pyrrole-2,5-dione), AR-AO144-18 (1-[(4-methoxyphenyl)methyl]-3-(5-nitro-1,3-thiazol-2-yl)urea), CT99021, CT20026, BIO ((2Z, 3′E)-6-bromoindirubin-3′-oxime), BIO-acetoxime, pyridocarbazole-cyclopentadienylruthenium complex, OTDZT, α-4-dibromoacetophenone and lithium. These may also be used in combinations of two or more. Antisense oligonucleotides or siRNA for mRNA of GSK3β, antibodies that bind GSK3β, or dominant negative GSK3β variants may also be used as GSK3β inhibitors, and are either commercially available or can be synthesized according to publicly known methods.

In step 1) of the production method of the present invention, the concentration of the GSK3β inhibitor in the medium may be appropriately adjusted depending on the type of GSK3p inhibitor added, and it may be 0.01 to 20 μM, for example, and preferably 0.1 to 10 μM. When CHIR99021 is used, its concentration in the medium will typically be 0.1 to 1 μM, preferably 0.5 to 1 μM and more preferably 1 μM.

The culturing period for step 1) of the production method of the present invention is not particularly restricted so long as it is a time period allowing the desired cells to be obtained, but it will typically be 6 to 14 days, preferably 8 to 12 days and more preferably 9 to 11 days (especially 10 days).

The cell culture density is not particularly restricted so long as the cells can proliferate. It will typically be 1.0×10¹ to 1.0×10⁶ cells/cm², preferably 1.0×10² to 1.0×10⁵ cells/cm², more preferably 1.0×10³ to 1.0×10⁴ cells/cm², and more preferably 3.0×10³ to 1.0×10⁴ cells/cm² (especially 3.6×10³ cells/cm²).

Before step 1) of the production method of the present invention, the cells may be cultured in medium containing neither TGFβ inhibitor nor GSK3β inhibitor, with no particular restrictions on the culturing time so long as it is a period allowing the desired cell count to be obtained. This will typically be 2 to 6 days. Such culturing is preferably carried out under feeder-free and xeno-free conditions.

Step 1) of the production method of the present invention can yield neural crest cells maintaining their multipotency. Such neural crest cells have the properties of neural crest cells (i.e. expressing TFAP2A, and also expressing one or more genes selected from among SOX10, PAX3, NGFR, CDH6, TWIST, DLX1 and CDH11), while also expressing SOX10, PAX3, NGFR and CDH6 and not expressing TWIST and/or DLX1. According to one aspect, the neural crest cells maintaining multipotency have the property of expressing TFAP2A, SOX10, PAX3, NGFR and CDH6 and not expressing TWIST, DLX1 or CDH11. The term “maintaining multipotency” here means having differentiation potency to neurons, glial cells and mesenchymal stem cells, and preferably also having differentiation potency to osteocytes, chondrocytes and melanocytes.

The neural crest cells obtained by step 1) of the production method of the present invention may be supplied to step 2) after sorting by FACS (Fluorescence-Activated Cell Sorting), etc. using high CD271 expression as the marker.

The ALK inhibitor used in step 2) of the production method of the present invention may be any of those mentioned above (which can be used in step 1)), without any particular restrictions. Preferred ALK inhibitors are one or more selected from the group consisting of SB431542, A83-01, LDN193189, GW788388, SM16, IN-1130, GW6604 and SB505124. A particularly preferred TGFβ inhibitor is SB431542. These may also be used in combinations of two or more.

In step 2) of the production method of the present invention, the concentration of the ALK inhibitor in the medium may be appropriately adjusted depending on the type of ALK inhibitor added, but it will typically be 1 to 50 μM, preferably 2 to 40 μM and more preferably 5 to 20 μM. When SB431542 (4-[4-(1,3-benzodioxol-5-yl)-5-(2-pyridinyl)-1H-imidazol-2-yl]-benzamide) is used, its concentration in the medium will typically be 1 to 40 μM, preferably 5 to 20 μM and more preferably 10 μM.

The EGF (Epidermal Growth Factor) used may be the one marketed by FujiFilm-Wako Pure Chemical Industries, etc. The concentration of EGF in the medium is not particularly restricted but will typically be 5 to 100 ng/ml, preferably 20 to 40 ng/ml and more preferably 20 ng/ml.

The FGF (Fibroblast Growth Factors) used may also be those marketed by FujiFilm-Wako Pure Chemical Industries, etc. FGF of 22 different types are known in humans, with bFGF (basic fibroblast growth factor, also known as FGF-2) being preferred. The concentration of FGF in the medium is not particularly restricted but will typically be 10 to 200 ng/ml, preferably 20 to 40 ng/ml and more preferably 20 ng/ml.

As used herein, the description “culture medium containing substantially no GSK-3β inhibitor” may mean not only that GSK-3β is not detected in the culture medium but also that the culture medium contains no GSK3β inhibitor at a higher concentration than the concentration that exhibits the same effect as 10 nM CHIR99021.

That an effect is the same as the effect exhibited by 10 nM CHIR99021 can be evaluated based on GSK3β inhibiting activity. GSK3β inhibiting activity can be measured using the gene expression regulating function of GSK3β in the Wnt/β-catenin pathway (specifically, the β-catenin-phosphorylating function), as the marker. Specifically, this can be done by the method established in Example 9 of PTL 1. This will now be explained in detail.

In the Wnt/β-catenin pathway, GSK3β functions to phosphorylate β-catenin in the absence of Wnt-ligand. Phosphorylated β-catenin is ubiquitinated and degraded in the proteasome, resulting in suppression of gene expression downstream from the Wnt-p-catenin pathway. When GSK3β is inhibited in this pathway, β-catenin is not degraded but migrates into the nucleus, inducing gene expression downstream from the Wnt-p-catenin pathway, together with other transcription factors such as T-Cell Factor (TCF)/Lymphoid Enhancer Factor (LEF). A CellSensor LEF/TCF-bla HCT-116 Cell Line (Thermo Fisher, K1676) has LEF/TCF incorporated in a stably expressing manner, with a reporter gene (beta-lactamase reporter gene) incorporated so as to be expressed under the control of LEF/TCF. Expression of the reporter gene in the cell line in the absence of Wnt-ligand is a marker of inhibition of GSK3β function (β-catenin phosphorylating function). An assay of the same cell line can measure the GSK3β inhibiting activity of a GSK3β inhibitor.

The assay is carried out according to the Invitrogen protocol (CellSensor (registered trademark) LEF/TCF-bla HCT 116 Cell-based Assay Protocol). Specifically, the LEF/TCF-bla HCT-116 cells are suspended in assay medium (OPTI-MEM, 0.5% dialyzed FBS, 0.1 mM NEAA, 1 mM sodium pyruvate, 100 U/mL/100 μg/mL, Pen/Strep) (312,500 cells/mL). The cell suspension is seeded in each well of an assay plate (10,000 cells/well) and cultured for 16-24 hours. CHIR99021 is added to each well (10 nM concentration), and culturing is continued for 5 hours. A beta-lactamase substrate solution (LiveBLAzer-FRET B/G (CCF4-AM) Substrate Mixture) is added to each well (8 μL/well), and the mixture is incubated for 2 hours. The fluorescence value is measured with a fluorescent plate reader. The measurement is conducted for two wells, calculating the average which is used as the effect exhibited by 10 nM CHIR99021.

For a GSK3β inhibitor other than CHIR99021 (i.e. the GSK3β inhibitor to be evaluated), the fluorescence value is measured under different concentration conditions (0.316, 1.00, 3.16, 10.0, 31.6, 100, 316, 1000, 3160 and 10,000 nM) by the experiment system, and the concentration at which GSK3β inhibiting activity exhibits the same GSK3β inhibiting activity as 10 nM CHIR99021 can be determined by drawing a calibration curve according to an established method. When the fluorescence value is equal to background level, GSK-3β may be evaluated as being below detection sensitivity in the culture medium.

The culturing period for step 2) of the production method of the present invention is not particularly restricted so long as it is a time period allowing the desired cells to be obtained, but it will typically be 7 to 45 days, preferably 10 to 30 days and more preferably 14 to 21 days. As shown in the following Examples, step 2) of the production method of the present invention can maintain NCCs with differentiation potency to mesenchymal stromal cells, for prolonged periods (45 days or longer). The culturing period may therefore be 45 days or longer.

The cell culture density is not particularly restricted so long as the cells can proliferate. It will typically be 1.0×10¹ to 1.0×10⁶ cells/cm², preferably 1.0×10² to 1.0×10⁵ cells/cm², more preferably 1.0×10³ to 1.0×10³ cells/cm², and more preferably 5.0×10³ to 5.0×10⁴ cells/cm² (especially 1.0×10⁴ cells/cm²).

In step 1) and step 2) of the production method of the present invention, the cells are cultured under feeder-free and xeno-free conditions. Preferably all of the steps in the production method of the present invention are carried out under feeder-free and xeno-free conditions. As used herein, “feeder-free” means medium or culturing conditions without other cell types (i.e., feeder cells) with an assisting role that are used to prepare culturing conditions for the cells to be cultured. Also, “xeno-free” means medium or culturing conditions without components derived from a different organism than the species of the cells to be cultured.

The feeder-free and xeno-free medium to be used for the present invention is not particularly restricted and may be StemFit (registered trademark) AK02 medium (Ajinomoto Co., Inc.), StemFit (registered trademark) AK03 medium (Ajinomoto Co., Inc.), StemFit (registered trademark) Basic03 medium, CTS (registered trademark) KnockOut SR XenoFree Medium (Gibco), mTeSR1 medium, TeSRI medium (Stem Cell Technologies), Iscove's modified Dulbecco's medium (GE Healthcare) and so on. AK03 medium is preferred among these.

If necessary, the medium may contain other substances including one or more serum substitutes such as Knockout Serum Replacement (KSR), N2 supplement (Invitrogen), B27 supplement (Invitrogen), albumin, transferrin, apotransferrin, fatty acids, insulin, collagen precursors, trace elements, 2-mercaptoethanol and 3′-thiolglycerol, and it may also contain one or more substances such as lipids, amino acids, L-glutamine, Glutamax (Invitrogen), non-essential amino acids, vitamins, growth factors, low molecular compounds, antibiotics, antioxidants, pyruvic acid, buffering agents, inorganic salts, selenic acid, progesterone and putrescine.

The culturing in steps 1) and 2) of the production method of the present invention may be by either suspension culture or adhesion culture, so long as the desired cells can proliferate, but preferably the culturing in both steps 1) and 2) is by adhesion culture. As used herein, “suspension culture” is culturing carried out under conditions that maintain the state in which the cells or cell aggregates are suspended in the culture medium, or in other words, conditions such that no rigid cell-substratum junction is formed between the cells or cell aggregates and the culture vessel. Also, “adhesion culture” is culturing under conditions such that a rigid cell-substratum junction is formed between the cells or cell aggregates and the culturing instrument.

The culture vessel used for adhesion culture may be one where the surface of the culture vessel is artificially treated to improve adhesion with cells (for example, coating treatment with extracellular matrix components such as basal membrane preparation, fibronectin or laminin or its fragments, entactin, collagen, gelatin Synthemax or vitronectin, or a polymer such as polylysine or polyornithine, or surface processing by positive charge treatment). A culture vessel coated with fibronectin is preferred.

Laminin or its fragments to be used for the present invention include laminin-111 or its fragment containing the E8 domain, laminin-211 or its fragment containing the E8 domain (e.g. iMatrix-211), laminin-121 or its fragment containing the E8 domain, laminin-221 or its fragment containing the E8 domain, laminin-332 or its fragment containing the E8 domain, laminin-3A11 or its fragment containing the E8 domain, laminin-411 or its fragment containing the E8 domain (e.g. iMatrix-411), laminin-421 or its fragment containing the E8 domain, laminin-511 or its fragment containing the E8 domain (e.g. iMatrix-511, iMatrix-511 silk), laminin-521 or its fragment containing the E8 domain, laminin-213 or its fragment containing the E8 domain, laminin-423 or its fragment containing the E8 domain, laminin-523 or its fragment containing the E8 domain, laminin-212/222 or its fragment containing the E8 domain, laminin-522 or its fragment containing the E8 domain, and so on. Laminin-511 or its fragment containing the E8 domain is preferred among these.

The culture vessel used for suspension culture is not particularly restricted so long as it allows “suspension culturing”, which may be determined as appropriate by a person skilled in the art. Examples of such culture vessels include a flask, tissue culture flask, dish, petri dish, tissue culture dish, multidish, microplate, microwell plate, micropore, multiplate, multiwell plate, chamber slide, schale, tube, tray, culture bag or roller bottle. A bioreactor is another example of a container for suspension culture. These culture vessels are preferably non-adhesive to cells to allow suspension culture. The non-cell-adhesive culture vessel used may also be one where the surface of the culture vessel is not artificially treated to improve adhesion with cells (for example, coating treatment with extracellular matrix).

The culturing temperature is not particularly restricted but may be about 30 to 40° C. and preferably about 37° C., with the culturing being carried out in a CO₂-containing air atmosphere with a CO₂ concentration of preferably about 2 to 5%.

Step 2) of the production method of the present invention can produce neural crest cells specialized for differentiation into a mesenchymal lineage. Another aspect of the present invention provides neural crest cells obtained by the production method of the present invention. Such neural crest cells typically have the properties of neural crest cells (i.e. expressing TFAP2A, and also expressing one or more genes selected from among SOX10, PAX3, NGFR, CDH6, TWIST, DLX1 and CDH11), while also having the property of expressing one or more genes selected from among TWIST, DLX1 and CDH11 and not expressing PAX3 and/or SOX10. According to one aspect, the neural crest cells specialized for differentiation into a mesenchymal lineage have the property of expressing TFAP2A, TWIST, DLX1 and CDH11 but not expressing PAX3 or SOX10.

Another aspect of the present invention, therefore, provides neural crest cells having all of the following properties (A) to (C) (hereunder also referred to as “neural crest cells of the present invention”).

(A) The cells are derived from pluripotent stem cells.

(B) The cells express one or more genes (and preferably all three genes) selected from among TWIST, DLX1 and CDH11.

(C) The cells do not express PAX3 and/or SOX10 (and preferably PAX3 or SOX10).

The neural crest cells of the present invention can also be read as cells having the following properties (A′) to (C′).

(A′) The cells are derived from pluripotent stem cells.

(B′) The cells express TFAP2A and one or more genes (and preferably all three genes) selected from among TWIST, DLX1 and CDH11.

(C′) The cells do not express PAX3 and/or SOX10 (and preferably PAX3 or SOX10).

The neural crest cells obtained by the production method of the present invention, or the neural crest cells of the present invention, may also be cells having all of the following properties (I) to (III).

(I) The cells are derived from pluripotent stem cells.

(II) The cells enhance expression of one or more genes (and preferably all three genes) selected from among TWIST, DLX1 and CDH11.

(III) The cells reduce or eliminate expression of one or more genes (and preferably all three genes) selected from among PAX3, CDH6 and SOX10.

In addition, the neural crest cells obtained by the production method of the present invention or the neural crest cells of the present invention can increase the survival rate and differentiation efficiency for inducing mesenchymal stem cells (by 2-fold or more, 4-fold or more or 10-fold or more), compared to neural crest cells before carrying out step 2) of the production method of the present invention.

As used herein, “enhance expression of a gene” means that the expression level of the gene is higher (such as 2-fold, 3-fold, 4-fold, 5-fold or higher), compared to the cells before carrying out step 2) of the production method of the present invention. Also “reduce or eliminate expression of a gene” means that the expression level of the gene is lower (such as ½, ⅕, 1/10 or lower), compared to the cells before carrying out step 2) of the production method of the present invention, or that no expression is observed.

As used herein, the phrase “specialized for differentiation into a mesenchymal lineage” means lacking (having lost) differentiation potency to nervous system cells and differentiation potency to melanocytes, while having (maintaining) differentiation potency to mesenchymal stem cells and through them to osteocytes, chondrocytes and adipocytes. Moreover, the phrase “a cell specialized for differentiation into a mesenchymal lineage” may be interpreted as “a cell with a tendency to easily differentiate into a mesenchymal lineage”.

According to another aspect, therefore, the neural crest cells obtained by the production method of the present invention or the neural crest cells of the present invention also have any one (and preferably all) of the following properties (D) to (F).

(D) The cells have differentiation potency to mesenchymal stem cells.

(E) The cells do not have differentiation potency to nervous system cells.

(F) The cells do not have differentiation potency to melanocytes.

As used herein, “do not have differentiation potency to nervous system cells” means that even when the cells are cultured for 3 weeks in neural differentiation-inducing medium (Neurobasal™ medium containing N2 supplement, B27 supplement, 2 mM L-glutamine, 10 ng/mL BDNF, 10 ng/mL GDNF, 10 ng/mL NT-3 and 10 ng/mL NGF), the cells do not differentiate into TuBB3 and peripherin-positive peripheral neurons, or GFAP-positive glial cells. For the present purpose, therefore, they “do not have differentiation potency to nervous system cells”, even if they differentiate into peripheral nerve cells or glial cells under culturing conditions different from those described above. Likewise, “do not have differentiation potency to melanocytes” means that even when the cells are cultured for 10 days in melanocyte differentiation-inducing medium (the medium StemFit (registered trademark) Basic03 containing 1 μM CHIR99021, 25 ng/mL BMP4 and 100 nM endothelin-3), they do not differentiate into MITF-positive melanocytes. Thus, even if they differentiate into melanocytes under culturing conditions different from those mentioned above, for the present purpose they “do not have differentiation potency to melanocytes”.

The description that a gene “expresses” or is “positive”, unless otherwise specified, is used herein in the sense that includes at least “production of mRNA encoded by the gene”, and preferably also “production of a protein encoded by the mRNA”. Therefore, the gene may be said to be expressed if production of mRNA encoded by the gene is detected at least by the following method (quantitative RT-PCR). Conversely, when production of mRNA encoded by the gene is not detected (i.e. below the detection limit) by the following method (quantitative RT-PCR), or when it is at background level, it can be said that the gene is not being expressed, or is negative. In other words, as used herein, when not detected by the following method the gene is not being expressed, even if production of mRNA is detected by a different method. The term “mRNA” as used herein also includes pre-mRNA. The method for detecting expression of a gene will now be explained in detail.

Total RNA is purified using an RNeasy Mini Kit (Qiagen Inc.) and treated with a DNase-one Kit (Qiagen Inc.) to remove the genomic DNA. A PrimeScript RT Master Mix (Takara) is used for reverse transcription of 500 ng of total RNA according to the manufacturer's protocol to obtain single-stranded cDNA. Quantitative PCR using Thunderbird SYBR qPCR Mix (Toyobo, Ltd.) is carried out using a QuantStudio 7 Flex Real-Time PCR System (Applied Biosystems).

### 2. Method for Producing Mesenchymal Stem Cells and Mesenchymal Cells

As mentioned above, neural crest cells obtained by the production method of the present invention or neural crest cells of the present invention have differentiation potency to mesenchymal stem cells. Therefore, another aspect of the present invention provides a method for producing mesenchymal stem cells comprising a step of culturing neural crest cells obtained by the production method of the present invention or neural crest cells of the present invention in mesenchymal stem cell differentiation-inducing medium (hereunder also referred to as “method for producing mesenchymal stem cells of the present invention”), as well as mesenchymal stem cells obtained by the method. As used herein, “mesenchymal stem cells” refers to cells having auto-replicating ability and having differentiation potency at least to osteocytes, chondrocytes and adipocytes. Preferably all of the steps in the method for producing mesenchymal stem cells of the present invention are carried out under feeder-free and xeno-free conditions.

The mesenchymal stem cell differentiation-inducing medium is not particularly restricted so long as it allows the neural crest cells obtained by the production method of the present invention or neural crest cells of the present invention to be induced to differentiate into mesenchymal stem cells by culturing. The mesenchymal stem cell differentiation-inducing medium used may be basal medium (such as αMEM medium) containing bovine serum, but it is preferably a commercially available xeno-free mesenchymal stem cell growth medium. Examples of commercially available xeno-free mesenchymal stem cell growth media include PRIME-XV MSC Expansion XSFM (FujiFilm-Wako Pure Chemical Industries), Cellartis (registered trademark) MSC Xeno-Free Culture Medium (Takara Bio, Inc.), MSC NutriStem (registered trademark) XF medium (Sartorius Stedim Japan) and so on.

The culturing period for the method for producing mesenchymal stem cells of the present invention is not particularly restricted so long as it is a time period allowing the desired cells to be obtained, but it will typically be 5 to 50 days, preferably 10 to 30 days and more preferably 14 days. After culturing, FACS analysis may be carried out for analysis of expression of cell surface antigens (such as CD73, CD44, CD45 and CD105), to confirm differentiation to mesenchymal stem cells.

The cell culture density is not particularly restricted so long as the cells can proliferate. It will typically be 1.0×10¹ to 1.0×10⁶ cells/cm², preferably 1.0×10² to 1.0×10⁵ cells/cm², more preferably 1.0×10³ to 1.0×10⁵ cells/cm², and more preferably 5.0×10³ to 5.0×10⁴ cells/cm² (especially 1.0×10⁴ cells/cm²).

For other culture conditions and culture methods, additives to the medium, specific examples of culture vessels, culture vessel surface treatment, and so on, the contents described above under “1. Method for producing neural crest cells specialized for differentiation into a mesenchymal lineage” are incorporated.

Yet another aspect of the present invention provides a method for producing mesenchymal cells (as differentiated cells) that includes a step of culturing mesenchymal stem cells obtained by the method for producing mesenchymal stem cells of the present invention in mesenchymal cell differentiation-inducing medium (hereunder also referred to as “method for producing mesenchymal cells of the present invention”), as well as mesenchymal cells obtained by the method. Examples of such mesenchymal cells include osteocytes, chondrocytes, adipocytes, and so on. Preferably all of the steps in the method for producing mesenchymal cells of the present invention are carried out under feeder-free and xeno-free conditions.

The osteocyte differentiation-inducing medium includes basal medium (such as, αMEM) containing 10% FBS, 0.1 μM dexa-methasone, 50 μg/ml ascorbic acid and 10 mM β-glycerophosphate, commercially available xeno-free osteocyte differentiation-inducing medium (such as MSCgo™ Rapid Osteogenic Differentiation Medium (Biological Industries)), and so on. The method for differentiation into osteocytes may be, specifically, a method of seeding 4×10⁴ mesenchymal stem cells in a gelatin-coated 12-well plate and culturing for 30 days in the aforementioned osteocyte differentiation-inducing medium. Calcified nodules are detected by Alizarin Red staining to confirm differentiation to osteocytes.

Chondrocyte differentiation-inducing medium includes basal medium (such as DMEM/F12) containing 1% (v/v) ITS+Premix, 0.17 mM AA2P, 0.35 mM proline, 0.1 mM dexamethasone, 0.15% (v/v) glucose, 1 mM sodium pyruvate, 2 mM GlutaMAX, 40 ng/ml PDGF-BB, 100 ng/mL TGF-β3, 10 ng/ml BMP4 and 1% (v/v) FBS, commercially available xeno-free chondrocyte differentiation-inducing medium (such as MSCgo™ Chondrogenic XF (Biological Industries)), and so on. The method for differentiation into chondrocytes may be, specifically, a method of spotting 5 μl of mesenchymal stem cell suspension in a fibronectin-coated plate and culturing for 1 hour, and then adding 1 ml of the aforementioned differentiation-inducing medium after 1 hour and culturing for 14 days. Differentiation to chondrocytes can be confirmed by Alcian Blue staining.

Adipocyte differentiation-inducing medium includes basal medium containing 60 μM indomethacin, 0.5 mM IBMX and 0.5 μM hydrocortisone, commercially available xeno-free adipocyte differentiation-inducing medium (for example, hMSC—Human Mesenchymal Stem Cell Adipogenic Differentiation Medium (Lonza), or MSCgo™ Adipogenic XF (Biological Industries), and so on. The method for differentiation into adipocytes may be, specifically, a method of seeding 4×10⁴ mesenchymal stem cells in a gelatin-coated plate and culturing for 32 days in the aforementioned osteocyte differentiation-inducing medium. Differentiation to adipocytes can be confirmed by Oil red O staining.

For other culture conditions and culture methods, additives to the medium, specific examples of culture vessels, culture vessel surface treatment, and so on, the contents described above under “1. Method for producing neural crest cells specialized for differentiation into a mesenchymal lineage” are incorporated.

### 3. Agent for Cell Transplantation Therapy

When transplanted into sites of bone, cartilage or muscle loss, mesenchymal stem cells obtained by the method for producing mesenchymal stem cells of the present invention, and mesenchymal cells (as differentiated cells) obtained by the method for producing mesenchymal cells of the present invention (hereunder also collectively referred to as “mesenchymal cells of the present invention”) can exhibit superior repair ability compared to cells obtained by conventional methods. The mesenchymal cells of the present invention can therefore be suitably used for cell transplantation therapy. Another aspect of the present invention therefore provides an agent for cell transplantation therapy (hereunder also referred to as “agent for cell transplantation therapy of the present invention”) comprising mesenchymal cells of the present invention. The present invention also encompasses a method for treating damage (including loss) or disease of tissue (such as bone tissue, cartilage tissue, adipose tissue or muscular tissue (such as skeletal muscle tissue)), in which an effective amount of mesenchymal cells of the present invention is administered or transplanted into a mammal to be treated (such as a human, mouse, rat, monkey, cow, horse, pig or dog). The concept of “treating tissue damage” also includes regeneration of damaged tissue.

The purpose of transplanting mesenchymal cells of the present invention into the body may be for direct regeneration of damaged tissue, or for an indirect effect (such as a paracrine effect) due to factors secreted by the mesenchymal cells of the present invention. For example, the mesenchymal stem cells can exhibit therapeutic effects in patients with acute myocardial infarction, stroke, multiple system atrophy (MSA), graft-versus-host disease, Crohn disease, ischemic cardiomyopathy, spinal cord injury and so on.

When mesenchymal cells of the present invention are to be used for cell transplantation therapy, it is preferred to use cells derived from iPS cells established from somatic cells having the same or essentially the same HLA genotype as the recipient individual to be grafted, from the viewpoint of avoiding rejection reaction. Here, “essentially the same” means that the HLA genotype matches to a degree that allows immunoreaction against the grafted cells to be reduced by immunosuppressive agents, and examples are somatic cells having an HLA type that matches at the 3 gene loci HLA-A, HLA-B and HLA-DR, or 4 gene loci further including HLA-C. When a sufficient number of cells cannot be obtained due to age or constitution, they may be grafted in a state embedded in capsules or a porous container of polyethylene glycol or silicone, to avoid rejection reaction.

The mesenchymal cells of the present invention may be combined with a pharmaceutically acceptable carrier by conventional means to produce a parenteral dosage form such as an injection, suspending agent or intravenous drip. One aspect of the present invention therefore provides a method for producing an agent for cell transplantation therapy which includes a step of formulating mesenchymal cells of the present invention. The method may also include a step of preparing the mesenchymal cells of the present invention. The method may further include a step of storing the mesenchymal cells of the present invention.

Examples of pharmaceutically acceptable carriers that may be included in parenteral dosage forms include aqueous solutions for injection in physiological saline, isotonic solutions including glucose or other adjuvants (such as D-sorbitol, D-mannitol and sodium chloride), and so on. The agent for cell transplantation therapy of the present invention may also include a buffering agent (such as phosphate buffer or sodium acetate buffer), a soothing agent (such as benzalkonium chloride or procaine hydrochloride), a stabilizer (such as human serum albumin or polyethylene glycol), a preservative or an antioxidant. When the agent for cell transplantation therapy of the present invention is to be formulated as an aqueous suspension, the cells may be suspended in the aqueous solution at 1×10⁶ to about 1×10⁸ cells/mL, for example. The dosage or grafting amount of the mesenchymal cells of the present invention or their pharmaceutical composition, and the number of administrations or grafts, may be appropriately determined depending on the age, body weight and symptoms of the mammal to which it is to be administered.

The agent for cell transplantation therapy of the present invention may also be provided in a cryopreserved state under normal conditions used for cryopreservation of cells, and thawed at the time of use. In this case, it may further comprise serum or a serum substitute, an organic solvent (such as DMSO) and so on. The concentration for serum or a serum substitute is not particularly restricted but may be about 1 to about 30% (v/v), and preferably about 5 to about 20% (v/v). The concentration for an organic solvent is also not particularly restricted but may be about 0 to about 50% (v/v), and preferably about 5 to about 20% (v/v).

The present invention will now be described in greater detail by Examples, with the understanding that the present invention is not in any way limited to these Examples.

### EXAMPLES

### Materials and Methods

(Human iPSCs and Human Primary Cultured Cell Lines)

Human iPSCs (1231A3, 1381A5, 1381B5, 1383D2, and 1383D10) were cultured on an iMatrix-511 (Nippi, Tokyo, Japan)-coated cell culture plate or dish in StemFit AK03N (Ajinomoto, Tokyo, Japan), as described above. The medium was changed every 2 days. Human mesenchymal stem/stromal cells (hAC-MSCs, hBM-MSCs, hUC-MSCs) were obtained from PromoCell (Heidelberg, Germany). MSCs were cultured on fibronectin (Millipore, Bedford, CA, USA)-coated culture dishes in PRIME-XV MSC Expansion XSFM medium (FUJIFILM Irvine Scientific, Tokyo, Japan). The medium was changed every 3 days. Human dermal fibroblasts (HDFs) were obtained from Cell Applications (San Diego, CA, USA) and cultured in Dulbecco's modified Eagle's medium (DMEM) (Thermo Fisher Scientific, Waltham, MA, USA) supplemented with 10% FBS (Thermo Fisher Scientific). The medium was changed every 3 days.

(Induction of Neural Crest Cells (NCCs) from iPSCs)

Human iPSCs were seeded onto iMatrix-511-coated plates or dishes at a density of 3.6×10³ cells/cm² in StemFit AK03N medium and maintained in culture for 4 days. For NCC induction, the cells were cultured in StemFit Basic03 (equivalent to AK03N without bFGF, Ajinomoto, Tokyo, Japan) with 10 μM SB431542 (FUJIFILM Wako) and 1 μM CHIR99021 (Axon Medchem, Reston, VA, USA) for 10 days. Cell numbers were counted using a Countess II FL (Thermo Fisher Scientific). The medium was changed every 2 days from day 0 to 6 and every day from day 7 to 10.

### (Differentiation of NCCs)

For peripheral neuron and glial cell differentiation, 1×10⁵ CD271^{high} sorted NCCs were seeded onto fibronectin-coated 12-well plates and cultured in Neurobasal (Thermo Fisher Scientific) medium supplemented with B27 supplement (Thermo Fisher Scientific), N-2 supplement (Thermo Fisher Scientific), 2 mM L-glutamine (FUJIFILM Wako, Tokyo, Japan), 10 ng/mL BDNF (FUJIFILM Wako), GDNF (FUJIFILM Wako), NT-3 (FUJIFILM Wako), and NGF (FUJIFILM Wako) for 3 weeks. The medium was changed every 3 days. Differentiation was confirmed by immunostaining with peripherin, TUBB3, and GFAP.

For melanocyte differentiation, 2.5×10⁵ CD271^{high} sorted NCCs were seeded onto fibronectin-coated 12-well plates and cultured in Basic03 supplemented with 1 μM CHIR99021, 25 ng/mL BMP4 (R&D Systems, Minneapolis, MN, USA), and 100 nM Endothelin-3 (TOCRIS, Bristol, UK) for 10 days. The medium was changed every 2 days. Differentiation was confirmed by immunostaining with MITF.

### (NCC Expansion Culture)

CD271^{high} sorted NCCs were seeded onto fibronectin-coated plates at a density of 1×10⁴ cells/cm² in Basic03 supplemented with 10 μM SB431542, 20 ng/mL EGF, and FGF2. The medium was changed every 3 days. For passaging, cells were dissociated with Accutase (Innovative Cell Technologies, San Diego, CA, USA) and re-plated onto fibronectin-coated plates at a density of 1×10⁴ cells/cm². For preparing the frozen stock of NCCs, 5×10⁵ NCCs were suspended in 500 μl STEM-CELL BANKER GMP grade (Takara, Kusatsu, Japan) and frozen using CoolCell Cell Freezing Containers (Biocision, Kyoto, Japan).

(Induction of Mesenchymal Stromal Cells (XF-iMSCs) from NCCs)

Expanded NCCs (4 passages; PN4) were seeded onto fibronectin-coated plates at a density of 1×10⁴ cells/cm² in Basic03 supplemented with 10 μM SB431542, 20 ng/mL EGF, and FGF2. The medium was replaced the next day with PRIME-XV MSC Expansion XSFM medium. The morphology of cells started to change approximately 4 days after induction. Passages were performed every 4 days using Accutase at a density of 1×10⁴ cells/cm². Human MSC markers (CD44, CD73, CD90, and CD105) were analyzed by FACS 14 days after the MSC induction.

### (Differentiation of XF-iMSCs)

For chondrogenic differentiation, 1.5×10⁵ XF-iMSCs were suspended in 5 μl of chondrogenic medium (DMEM/F12, Thermo Fisher Scientific), 1% (v/v) ITS+premix (Corning, Corning, NY, USA), 0.17 mM AA2P (Sigma, St. Louis, MO, USA), 0.35 mM Proline (Sigma), 0.1 mM dexamethasone (Sigma), 0.15% (v/v) glucose (Sigma), 1 mM sodium-pyruvate (Thermo Fisher Scientific), 2 mM GlutaMAX (Thermo Fisher Scientific), and 0.05 mM MTG (Sigma) supplemented with 40 ng/mL PDGF-BB (PeproTech, Rocky Hill, NJ, USA), 100 ng/mL TGF-β3 (R&D), 10 ng/mL BMP4, and 1% (v/v) FBS (Thermo Fisher Scientific) and were subsequently transferred to fibronectin-coated 24-well plates. A total of 1 mL of chondrogenic medium was added after 1 h. The cells were cultured for 14 days.

The differentiation properties of the cells were confirmed by Alcian blue staining. Briefly, induced cells were fixed for 30 min with 4% paraformaldehyde (PFA) (FUJIFILM Wako) and rinsed with phosphate buffered saline (PBS). These cells were then stained with Alcian Blue solution (1% Alcian Blue (MUTO PURE CHEMICAL CO., LTD, Tokyo, Japan) for 1 h at 25° C. For osteogenic differentiation, 4×10⁴ XF-iMSCs were seeded onto 0.1% gelatin-coated 12-well plates and cultured in MSCgo Rapid Osteogenic Differentiation Medium (Biological Industries, Cromwell, CT, USA) for 30 days. The medium was changed every 3 days. Differentiation properties were confirmed by the formation of calcified nodules, as detected with Alizarin Red (Merck, Darmstadt, Germany) staining. Briefly, culture wells were washed twice in PBS and fixed for 10 min at room temperature in 100% ethyl alcohol. The Alizarin Red solution (40 mM, pH 4.2) was applied to the wells for 10 min at room temperature. Nonspecific staining was removed by several washes with water. For adipogenic differentiation, 4×10⁴ XF-iMSCs were seeded onto a 0.1% gelatin-coated 12-well plate and cultured in hMSC Adipogenic Differentiation Medium (Lonza, Basel, Switzerland) for 32 days. The medium was changed every 3 days. Differentiation properties were confirmed by Oil Red O staining. The cells were fixed in 10% formalin for 1 h at room temperature, followed by incubation for 20 min in 0.3% Oil Red O staining solution (Sigma). Nonspecific staining was removed by performing several washes with water.

### (Immunocytochemistry)

Prior to performing immunostaining with antibodies, the cells on plates were fixed with 4% PFA/PBS (FUJIFILM Wako) at 4° C. for 15 min, washed twice with PBS, and incubated with 0.3% TritonX100 at 4° C. (as the surface-active agent for penetration processing) for 30 min, and any nonspecific binding was blocked with 3% BSA/PBS at 4° C. for 1 h. DAPI (1:1000; Thermo Fisher Scientific) was used to counterstain the nuclei. The primary antibodies used in this study are summarized in Table 1. Observations and assessments of samples were performed with BZ-X700 (Keyence, Osaka, Japan).

TABLE 1

| Antigen | [symbol] | [symbol] | [symbol] | [symbol] | [symbol] | [symbol] |
|---|---|---|---|---|---|---|
| SOX10 | goat | poly | — | SantaCruz | sc-17342 | 1/200 |
| CD271 | mouse | mono | — | ATS | AB-N07 | 1/500 |
| TFAP2A | mouse | mono | — | DSHB | 3B5 | 1/500 |
| TUBB3 | rabbit | poly | — | abcam | ab18207 | 1/5000 |
| Peripherin | mouse | mono | — | SantaCruz | sc-377093 | 1/200 |
| GFAP | rabbit | poly | — | abc am | ab7260 | 1/500 |
| MITF | rabbit | poly | — | Sigma | SAB4501879 | 1/200 |
| TWIST | rabbit | poly | — | Merck | ABD29 | 1/500 |
| DLX1 | mouse | mono | — | NOVUS | H00001745-M03 | 1/200 |
| hVimentin | rabbit | mono | — | abcam | ab16700 | 1/500 |
| Laminin-α2 | rat | mono | — | ALEXIS | ALX-804-190-C100 | 1/50 |
| MYH4 | mouse | mono | — | DSHB | BF-F3 | 1/50 |
| MYH3 | rabbit | poly | — | Sigma | HPA021808 | 1/200 |
| h-Lamin A/C | mouse | mono | — | SantaCruz | sc-7292 | 1/200 |
| Antigen | [symbol] | [symbol] | [symbol] | [symbol] | [symbol] | [symbol] |
| CD271 | mouse | mono | Alexa647 | BD Pharmingen | 560326 | 1/100 |
| CD44 | mouse | mono | APC | BD Pharmingen | 559942 | 1/100 |
| CD45 | mouse | mono | APC | BD Pharmingen | 560973 | 1/100 |
| CD73 | mouse | mono | APC | BD Pharmingen | 560847 | 1/100 |
| CD90 | mouse | mono | APC | BD Pharmingen | 559869 | 1/100 |
| CD105 | mouse | mono | APC | eBioscience | 17-1057 | 1/100 |
| HLA-DR | mouse | mono | APC | BD Pharmingen | 340549 | 1/100 |
| Mouse_IgG1_k | mouse | mono | Alexa647 | BD Pharmingen | 557714 | 1/100 |
| Mouse_IgG2b_k | mouse | mono | APC | BD Pharmingen | 555745 | 1/100 |
| Mouse_IgG1_k | mouse | mono | APC | BD Pharmingen | 555751 | 1/100 |
| Mouse_IgG2a_k | mouse | mono | APC | BD Pharmingen | 555576 | 1/100 |

[symbol]

indicates data missing or illegible when filed

### (FACS Sorting)

FACS was performed using an AriaII instrument (BD Biosciences, Franklin Lakes, NJ, USA) according to the manufacturer's protocol. The antibodies used are listed in Table 1. In all experiments, an isotype control was used to remove the nonspecific background signal.

### (Quantitative RT-PCR)

Total RNA was purified using the RNeasy Mini Kit (Qiagen, Valencia, CA, USA) and treated with the DNase-one Kit (Qiagen) to remove genomic DNA. 500 ng of total RNA was reverse transcribed to obtain single-stranded cDNA using PrimeScript RT Master Mix (Takara) according to the manufacturer's instructions. Quantitative PCR with the Thunderbird SYBR qPCR Mix (TOYOBO, Osaka, Japan) was performed using the QuantStudio 7 Flex Real-Time PCR System (Applied Biosystems, Forester City, CA, USA) in triplicate. Primer sequences are listed in Table 2. Each sequence listed in Table 2 is shown in the sequence listing as SEQ ID NOs: 1 to 40.

TABLE 2

| | | |
|---|---|---|
| NGFR | CCGTTGGATTACACGGTCCA | GACAGGGATGAGGTTGTCGG |
| SOX10 | GAGCTGGACCGCACACCTTGGG | AACGCCCACCTCCTCGGACCTC |
| TFAP2A | AGGGCCTCGGTGAGATAGTT | AAGAGTTCACCGACCTGCTG |
| RHOB | ATCCCCGAGAAGTGGGTCC | CGAGGTAGTCGTAGGCTTGGA |
| PAX3 | CGGCATCCTGAGCGAGCGAG | ACTCGGGCCTCGGTGAGCTT |
| SNAI2 | TGTGACAAGGAATATGTGAGCC | TGAGCCCTCAGATTTGACCTG |
| CDH6 | CTGCGACGGATGCAGATGAT | CCCTGTTTTCTCGATCCATGTTG |
| PAX6 | CTGGCTAGCGAAAAGCAACAG | CCCGTTCAACATCCTTAGTTTATCA |
| TWIST | GTCCGCAGTCTTACGAGGAG | GCTTGAGGGTCTGAATCTTGCT |
| DLX1 | TGCCAGAAAGTCTCAACAGCC | CGAGTGTAAACAGTGCATGGA |
| CDH11 | AGAGAGCCCAGTACACGTTGA | TTGGCATGATAGGTCTCGTGC |
| POU5F1 | GACAGGGGGAGGGGAGGAGCTAGG | CTTCCCTCCAACCAGTTGCCCCAAAC |
| ALP | GCGGTGAACGAGAGAATG | CGTAGTTCTGCTCGTGCAC |
| OCN | GTGACGAGTTGGCTGACC | TGGAGAGGAGCAGAACTGG |
| RUNX2 | ACTACCAGCCACCGAGACCA | ACTGCTTGCAGCCTTAAATGACTC |
| BMP2 | CTGTATCGCAGGCACTCA | CTCCGTGGGGATAGAACTT |
| BMP4 | CACAGCACTGGTCTTGAGTATCCTG | CTCAGGGATGCTGCTGAGGTTAAAG |
| BMP7 | CCACCTGTAATCCCAGCACT | GAGAAAGACCAGAGGGTCCA |
| 18S | GTAACCCGTTGAACCCCATT | CCATCCAATCGGTAGTAGCG |
| ACTB | AGGTCTTTGCGGATGTCCACGT | CACCATTGGCAATGAGCGGTTC |

### (Transcriptome Analysis)

Total RNA was purified using the RNeasy Micro Kit (Qiagen) and treated using the DNase-one kit (Qiagen) to remove genomic DNA. 10 ng of total RNA was reverse transcribed to obtain single-stranded cDNA using the SuperScript VILO cDNA Synthesis Kit (Thermo Fisher Scientific). cDNA library synthesis for the Ion Ampliseq transcriptome was performed using the Ion AmpliSeq Transcriptome Human Gene Expression Core Panel (Thermo Fisher Scientific) and the Ion Ampliseq Library Kit Plus (Thermo Fisher Scientific) according to the manufacturer's protocol. Barcode-labeled cDNA libraries were analyzed using the Ion S5 XL System (Thermo Fisher Scientific) and the Ion 540 Chip Kit (Thermo Fisher Scientific).

### (Preparation of XF-C-iMSCs)

XF-C-iMSCs were prepared as reported previously, with minor modifications (Stem Cell Res Ther 8, 101 (2017)). Briefly, XF-iMSCs were seeded at a density of 1.0×10⁵ cells/well in 48-well plates (Corning, Corning, NY, USA) coated with fibronectin and cultured with Prime-XV MSC expansion XSFM for 4 days. To obtain XF-C-iMSCs, confluent cells that had formed on the cellular sheet consisting of the ECM produced by MSCs themselves were scratched using a micropipette tip and then torn off. The iMSC/ECM complexes detached from the bottom of the plate in a sheet shape were transferred to a 24-well ultra-low binding plate (Corning) and rolled up to form a round clump of cells. The cell clumps were maintained in Prime-XV MSC expansion XSFM or MSCgo Osteogenic differentiation medium (Biological Industries) for 2, 5, or 10 days.

### (Staining of XF-C-iMSCs)

XF-C-iMSCs were fixed with 4% PFA in PBS. The samples were embedded in paraffin, and 5 μm thick serial sections were prepared. The specimens were then stained with hematoxylin and eosin (H&E) or Alizarin Red S and observed using a Nikon Eclipse E600 microscope (Nikon, Kawasaki, Japan).

### (Surgical Procedures)

Male NOD/SCID mice (7 to 8-week-old) (Charles River Laboratories Japan) were employed as a calvarial defect model after ethical approval was obtained from the Animal Care Committee of Hiroshima University. Surgery was performed under general anesthesia, with an intraperitoneal injection of 20% ethyl carbamate (30 mg/kg body weight). The skin at the surgical site was shaved and disinfected, and a sagittal skin incision was made from the occipital to the frontal bone. The skin flap, including the periosteum, was then dissected and elevated. Avoiding the cranial suture, a 1.6-mm diameter defects was created in the parietal bone. XF-C-iMSCs cultured with MSCgo osteogenic differentiation medium for 2 days were transplanted into the defect with no artificial scaffold. In addition, the implantation of XF-C-BMMSCs maintained in the same medium was used as a control. The skin incision was then closed using 4-0 silk sutures.

### (Micro-CT Analysis)

Mice were sacrificed 28 days after surgery, and the cranial region was imaged using a SkyScan1176 in vivo micro-CT (Bruker, Billerica, MA, USA). Three-dimensional reconstructions were generated using the CTVOL software (Bruker). The volume of newly formed bone inside the bone defect was determined using CT-An software (Bruker).

### (Tissue Preparation and Histological Analysis of the Skull)

The mice were sacrificed 28 days after surgery. Calvarial bones were collected, fixed with 4% PFA overnight, and decalcified with 10% (v/v) ethylenediaminetetraacetic acid (pH 7.4) for 10 days. After decalcification, the specimens were dehydrated through graded ethanol, cleared with xylene, and embedded in paraffin. Serial sections (5 μm) were cut in the frontal plane. These sections representing the central portion of the bone defect were stained with H&E and observed using a Nikon Eclipse E600 microscope. For Azan staining, the slides were incubated in mordant solution (a mixture of equal parts of 10% potassium dichromate and 10% trichloroacetic acid) for 10 min and washed with H₂O.

The slides were then incubated in azocarmine G solution (0.1% azocarmine G, 1% acetic acid) for 30 min. The specimens were briefly washed with H₂O, differentiated with anilin alcohol (0.1 mL anilin dissolved in 100 mL 95% ethanol), washed with acetic alcohol and H₂O, incubated in phosphotungstic acid (5%) for 1 h, briefly washed with H₂O, washed in aniline blue/orange G solution (0.5% anilin blue, 2% orange G, 8% acetic acid) for 30 min, and then briefly washed with H₂O. Subsequently, the slides were incubated in 100% alcohol and xylene before they were embedded using mounting medium. To detect human vimentin expression in the tissue, immunofluorescence analysis was performed. Briefly, serial sections (20 μm) were blocked with 1% BSA/0.1% Triton-X/PBS blocking solution at room temperature for 30 min. These sections were then incubated with rabbit anti-human vimentin monoclonal IgG antibody (Abcam; #SP20) at 4° C. overnight. After washing with PBS three times for 5 min, the samples were incubated for 1 h with an Alexa Fluor 488 (registered trademark) goat anti-rabbit IgG antibody (Thermo Fisher Scientific) at room temperature. Nuclei were counterstained with DAPI. Fluorescence signals were detected using a Zeiss LSM 510 laser scanning confocal microscope (Zeiss, Oberkochen, Germany).

### (Skeletal Muscle Injury and Transplantation)

For the transplantation of XF-iMSCs, 8-to 16-week-old NSG mice were purchased from Charles River Japan (Yokohama, Japan). Mice were anesthetized with 3% Forane inhalant liquid (AbbVie, North Chicago, IL, USA). The midportion of the tibialis anterior muscle (TA muscle) was then continuously crushed by direct clamping with forceps for 1 min under constant pressure determined using the same pressure gauge (Biores Open Access 2, 295-306 (2013)). XF-iMSCs or human skin fibroblasts (HDFs) were suspended in αMEM (2×10⁵ cells/50 μL) and injected using a 27 G micro-syringe at the center of the injured sites of the TA muscles 24 h post injury.

### (Tissue Preparation and Histological Analysis of Muscle)

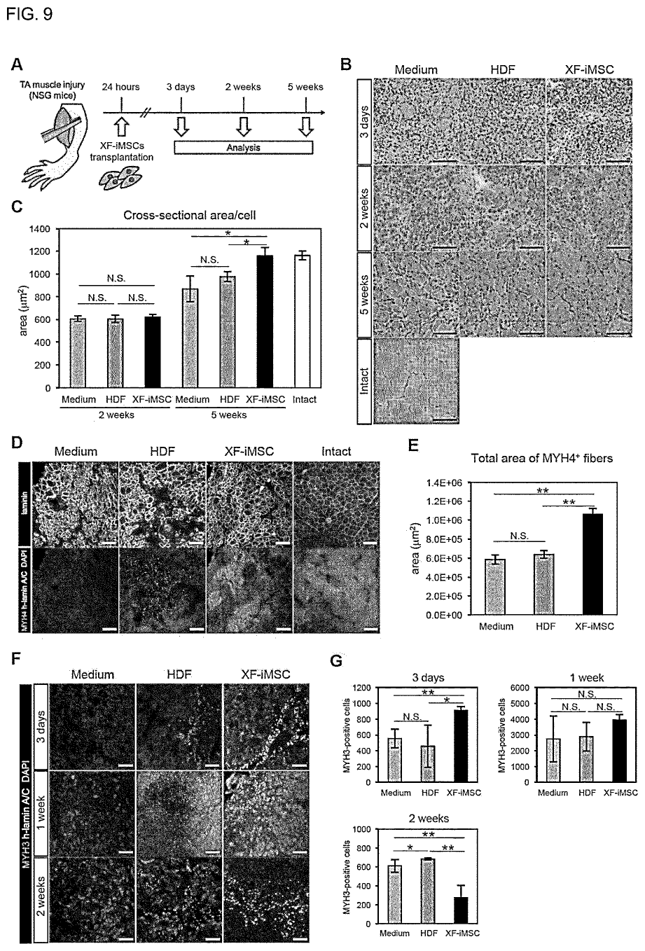

Mice were sacrificed 3 days, 2 weeks, and 5 weeks post injury. Tibialis anterior muscles were mounted in Tragacanth Gum (FUJIFILM Wako) and frozen with liquid nitrogen (PLoS One 8, e61540 (2013)). Serial sections (10 μm) were cut using a cryostat. Sections were stained with H&E and observed using an Olympus BX51 microscope (Olympus, Tokyo, Japan). Four sections prepared from the middle of each TA muscle samples were stained, and the entire transverse section of all sections was photographed and analyzed. Of the values obtained from the four thin sections taken, the one with the highest value was adopted as the data for each sample. Immunofluorescence images were acquired using the Zeiss LSM 710 laser scanning confocal microscope (Zeiss). Area measurement and cell counting were performed using Hybrid cell count software BZ-H3C (Keyence). In FIG. 9C, images stained with Laminin antibody were analyzed with KEYENCE image analysis software to calculate the average area of myofibers. In FIG. 9E, images stained with anti-MYH4 antibody were analyzed with KEYENCE image analysis software to calculate the area of the positive area. In FIG. 9G, images stained with anti-MYH3 antibody were counted for the number of positive fibers.

(Myotube Differentiation from Mouse Newborn Myoblasts)

Myoblasts were isolated from newborn C57BL/6 mice (CLEA Japan, Tokyo), as described previously (Stem Cell Res 30, 122-129 (2018)). Myoblasts were cultured on 10% Matrigel (Corning)-coated dishes in high glucose DMEM with 20% FCS, 10% horse serum, 0.5% chicken embryo extract, 2.5 ng/mL FGF2, 10 μg/mL gentamycin, 1% antibiotic-antimitotic, and 2.5 μg/mL plasmocin prophylaxis (Proliferation Medium: PM). For myotube differentiation, 1×10⁵ myoblasts were seeded onto 10% Matrigel-coated 24-well plates and cultured in high glucose DMEM with 5% horse serum and 1% antibiotic-antimitotic (Differentiation Medium: DM) for 3 days. XF-iMSC-cultured medium was obtained by incubating XF-iMSCs in DM medium for 48 h. Human PXDN recombinant protein (Abnova, Taipei, Taiwan) was used at 0.5 μM. Myotube movement was analyzed using the ImageJ software with the TPIV plugin (https://signaling.riken.jp/toolsfimagej-plugins/490/).

### (Data Availability)

RNA-seq data that support the results of the Examples of the present application have been deposited in the Gene Expression Omnibus (GEO) database with the following accession code GSE206048, GSE206128, GSE206172. Proteome data that support the findings of the present Examples have been registered in jPOSTrepo (Japan Proteome Standard Repository) database with the following accession code JPST001693.

### Example 1: Induction of NCCs from Human iPSCs Under Xeno-Free Conditions

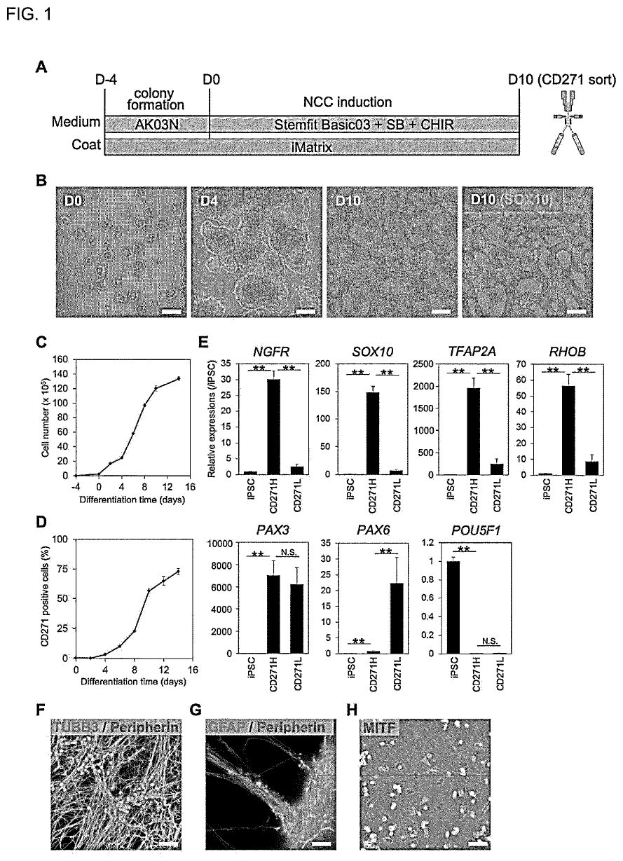

To induce NCCs from human iPSCs under xeno-free conditions, the previous induction protocol was modified. In the original protocol, iPSC lines maintained on an STO cell line transformed with neomycin resistance and LIF genes (SNL) stromal feeder cells in a culture dish coated with growth factor-reduced Matrigel, which was extracted from the Engelbreth-Holm-Swarm mouse were used. To avoid these animal components, a feeder-free and xeno-free iPSC line-1231A3 iPSCs were used and the cell line was maintained in iMatrix (laminin-511 E8 fragment)-coated culture dishes and in Stemfit AK03N xeno-free (defined and animal component-free) culture media (Sci Rep 4, 3594 (2014)) (FIG. 1A).

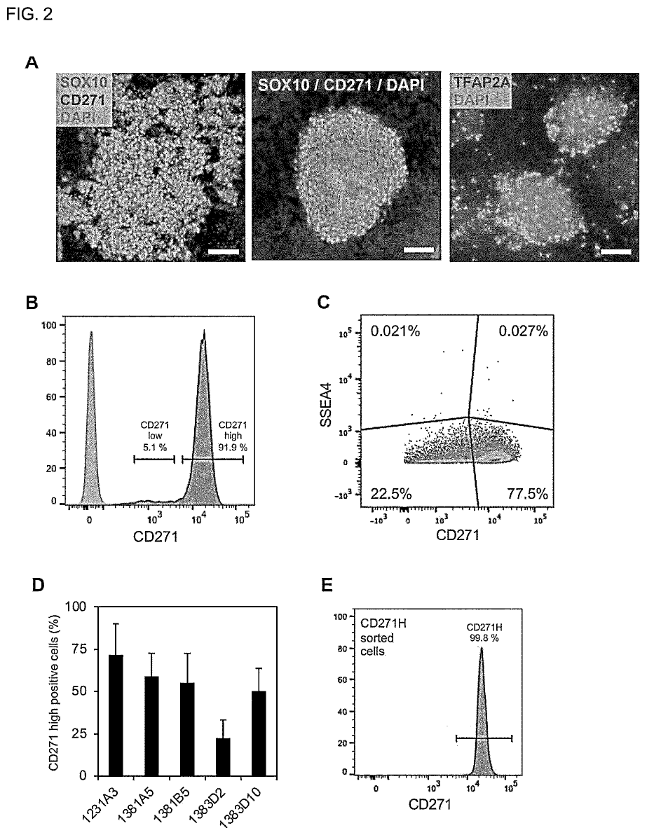

The iPSCs were cultured for 4 days until colonies formed, before starting the NCC induction. In the original protocol, bovine serum albumin (BSA) was used during the NCC induction. Here, BSA-containing basal medium was replaced with Stemfit Basic03, which is comparable to AK03N minus bFGF. Ten days after the NCC induction in Stemfit Basic03 medium supplemented with 10 μM SB431542 and 1 μM CHIR99021 (CHIR), the cells nearly reached confluence (FIG. 1B and C) in the cell-dense region and expressed the NCC markers SOX10, CD271, and TFAP2A (FIG. 1B and FIG. 2A). It was also confirmed that SOX10 and CD271, a cell-surface marker of NCC, mostly overlapped.

The induction efficiency was analyzed using fluorescence-activated cell sorting (FACS) with CD271 antibody, or the antibody and SSEA4 antibody. SSEA4-positive undifferentiated iPS cells were seldom detected (<0.05%) and the ratio of CD271^{high}-positive cells reached a peak of 90% (FIG. 2B, C). The robustness of this protocol was confirmed by repeated experiments using 1231A3 and other feeder-free and xeno-free iPSC lines (1231A3, 1381A5, 1381B5, 1383D2, and 1383D10; 71.8±18.3%, 59.0±13.7%, 55.2±17.5%, 15.0±11.1%, and 50.3±13.3%, respectively) (FIG. 2D).

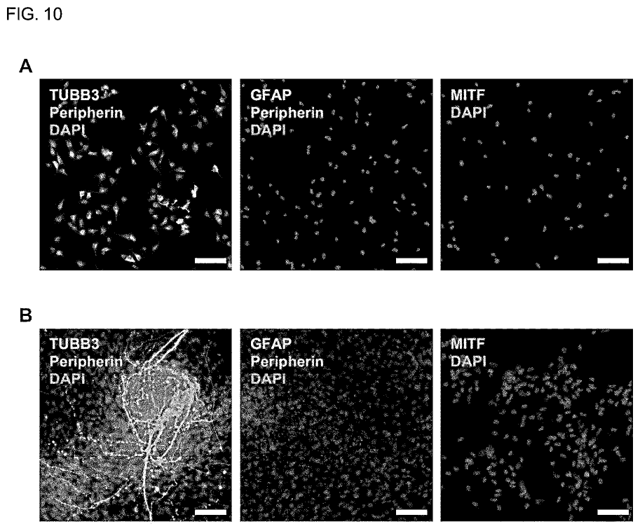

CD271^{high}-positive cells emerged until day 4 and gradually increased during induction (FIG. 1D). Since CD271 recognizes the cell-surface protein NGFR (p75), cell sorting with the CD271 antibody allowed for enrichment of NCCs (FIG. 2E). The enrichment was confirmed by the higher expression of NCC markers (NGFR, SOX10, TFAP2A, and RHOB) in CD271^{high} cells compared with that in the CD271^{low} cells (FIG. 1E). The expression of the NCC and neuron markers, PAX3 and PAX6, as well as the pluripotent cell marker POUF5F1 was also checked using quantitative real-time polymerase chain reaction (RT-qPCR). It was found that CD271^{low}-positive cells included neural cells (FIG. 1E). The multiple differentiation potential of the cells was confirmed by the induction of peripheral nervous system cells (TUBB3, Peripherin, and GFAP) (FIG. 1F and G) and melanocytes (MITF) (FIG. 1H). CD271^{high} cells were positive for TUBB3 and negative for Peripherin, GFAP and MITF (FIG. 10A). From CD271^{low} cells TUBB3-positive neurons were induced, but Peripherin-positive peripheral neurons were hardly detected, and MITF-positive pigment cells were not at all, consistent with the result that CD271^{low} cells included neuroectoderm (FIG. 10B).

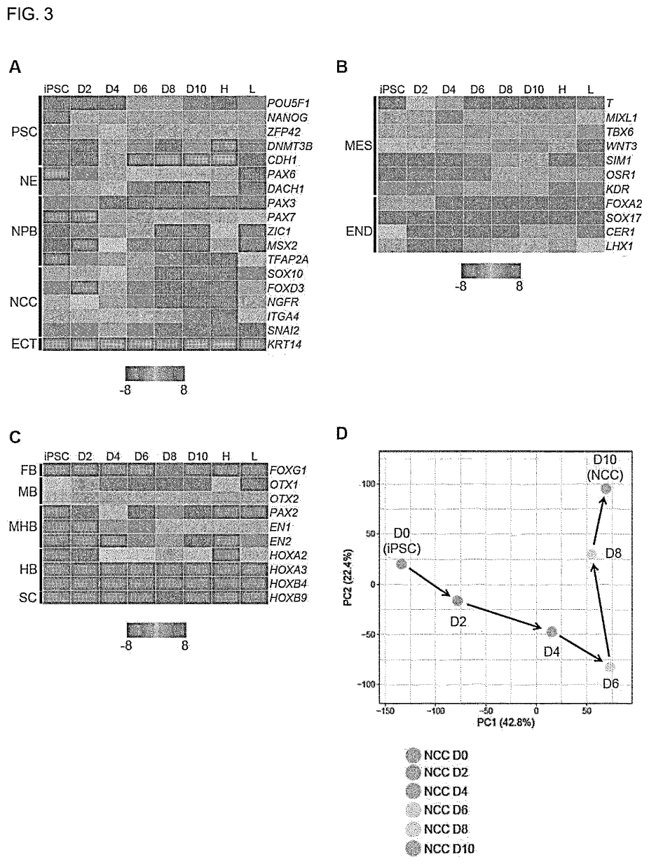

To investigate the developmental pathway followed by induced NCCs, the global gene expression profiles of NCCs on days 2, 4, 6, 8, and 10 post induction and those of CD271^{high} and CD271^{low} cells on day 10 were analyzed. During the first 6 days, the expression of pluripotent markers, such as POU5F1, NANOG, ZFP42, DNMT3B, and CDH1, was downregulated (FIG. 3A). Conversely, the expression of ectodermal markers, including neuroectoderm (PAX6 and DACH1), neural plate border (PAX3, PAX7, ZIC1, MSX2, and TFAP2A), and NCC (SOX10, FOXD3, NGFR, ITGA4, and SNAI2) markers, was upregulated. Consistent with the RT-qPCR data, CD271^{high} cells highly expressed NCC markers, whereas CD271^{low} cells highly expressed neuroectodermal markers.

These data confirm the successful enrichment of NCCs by CD271 FACS. The activation of an epidermal ectoderm marker (ECT), mesoderm markers (T, MIXL1, TBX6, WNT3, SIM1, OSR1, and KDR), and endoderm markers (FOXA2, SOX17, CER1, and LHX1) was marginal (FIG. 3B). The expression of region-specific homeobox genes revealed that the protocol of this Example induced cells in the midbrain and anterior hindbrain regions (positive for OTX1, OTX2, EN1, and HOXA2) but not in the forebrain or spinal cord (FIG. 3C). A principal component analysis (PCA) showed that a shift along primary component 1 (PC1, 42.8%) was observed during the first 6 days, whereas a major shift was observed along with PC2 (22.4%) from day 6 to day 10 (FIG. 3D). Taken together, these data suggest the directed differentiation of the midbrain and anterior hindbrain NCCs through the ectodermal lineage and dynamic fate restriction from pluripotency to neuroectoderm and NCCs during the first 6 days.

### Example 2: Verification of Neuronal Differentiation Potential of Xeno-Free NCC Expanded with TGFβ Inhibitor SB431542, EGF, and bFGF

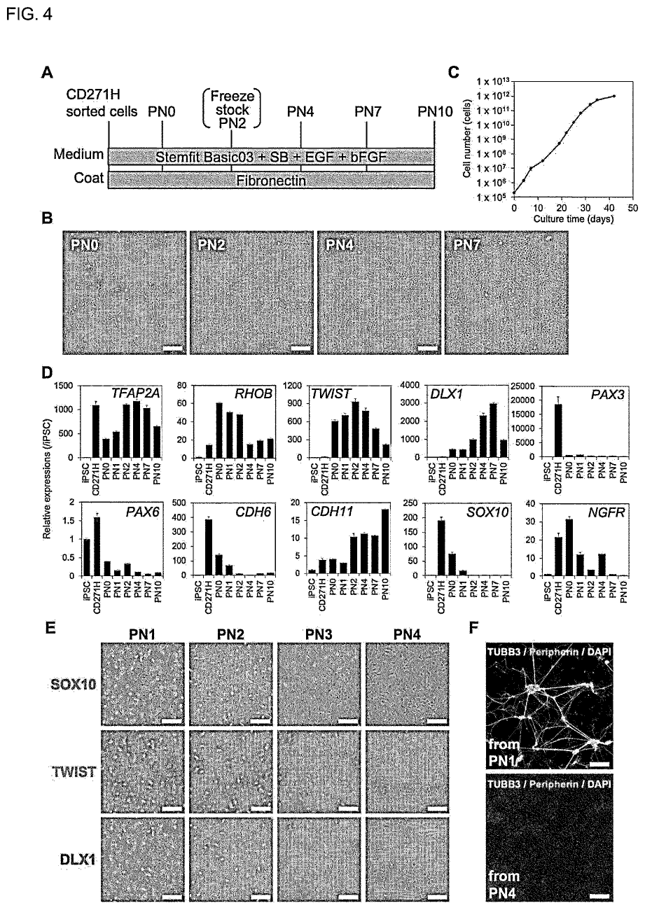

Previous reports have shown that induced NCCs can be expanded in a chemically defined medium (containing BSA) with the TGFβ inhibitor SB431542 (hereinafter referred to as “SB”), EGF, and bFGF on fibronectin-coated dishes (PLoS One 9, e112291 (2014), Exp Cell Res 316, 1148-1158 (2010)). Here, the present inventors also intended to investigate whether a xeno-free basal medium-Basic03-supplemented with SB, EGF, and bFGF could also expand NCCs (FIG. 4A). Under these conditions, cells proliferated and maintained a similar fibroblastic morphology for several passages (usually until around passage 7 (=30 days (until the number of days elapsed from the start of culture, the same applies hereafter))) (FIG. 4B and C). However, the proliferative speed decreased and eventually ceased.

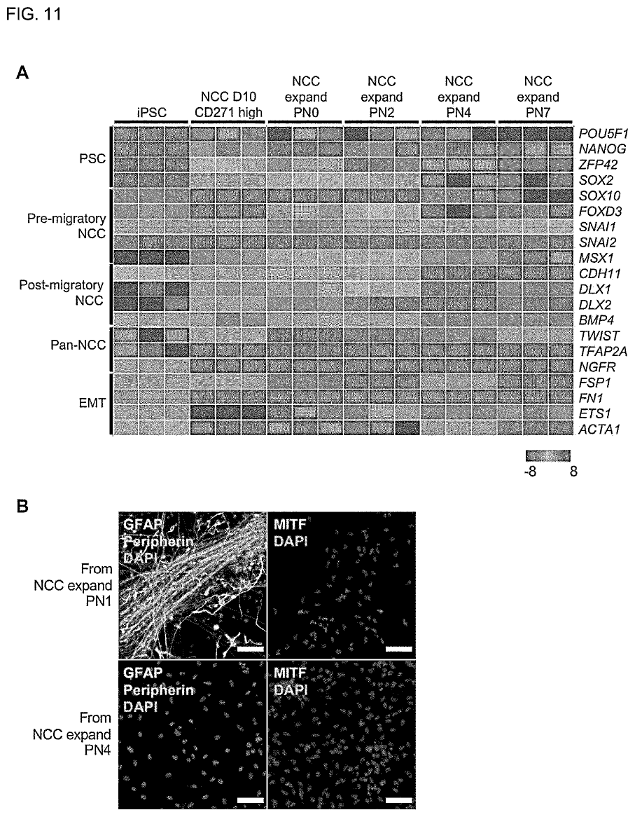

To check whether expanded cells maintained their NCC characteristics, the expression of NCC markers was analyzed (FIG. 4D). TFAP2A, a pan-NCC marker, was expressed in CD271^{high} cells and maintained until passage 10 (=45 days), suggesting that NCC characteristics were, at least in part, maintained during passages. The expression of RHOB, another NCC marker, was observed in CD271^{high} cells, peaked in early passages, and maintained until passage 10 (=45 days), also suggesting that NCC characteristics were maintained. The expression levels of TWIST and DLX1, markers of migrating NCCs, were low in CD271^{high} cells but expressed from passage 0 (=0-2 days) to passage 10 (=45 days), although they peaked at passage 2 (=7 days) and passage 7 (=30 days), respectively. PAX3, a marker of pre-migratory and early migrating NCCs, was expressed just after sorting, but its level significantly decreased after plating. The expression of PAX6, a marker of neuroectoderm, was low and comparative to that in iPSCs. These data suggest that the characteristics of NCCs changed gradually from pre-migratory to migratory during passage. In accordance with this idea, the switch of cadherin from CDH6 to CDH11 was observed with passaging. Strikingly, however, the expression of SOX10, a neural crest stem cell marker (Cell Stem Cell 15, 497-506 (2014)), was significantly downregulated during early passages (=2-7 days). NGFR, a pre-migratory and migratory NCC marker, was highly expressed in the early passages but hardly detected at passage 7 (=30 days). These data were also confirmed by a transcriptome analysis of markers for PSCs, pre-migratory NCCs, post-migratory NCCs, pan-NCCs and EMT (FIG. 11A) and immunocytochemistry with anti-SOX10, anti-TWIST, and anti-DLX1 antibodies (FIG. 4E). All data support the idea that cell characteristics change gradually with passaging.

DLX1 and CDH11 are markers for mesenchymal cells, and, since their expression increased at later passages, the present inventors hypothesized that expanded NCCs gained mesenchymal characteristics. To test this hypothesis, the differentiation properties of expanded NCCs were assessed by culturing NCCs under neuron, glia, melanocyte and MSC induction conditions. As expected, NCCs differentiated to form peripheral neurons and glia at passage 1 (=4 days) but not at passage 4 (=14 days) (FIG. 4F and FIG. 11B). Melanocytes were not differentiated from NCCs at passages 1 (=4 days) or 4 (=14 days) (FIG. 11B). These results suggested that NCCs cultured in expansion culture of the present invention, first lose their ability to differentiate into pigment cells and then into peripheral neurons and glia.

### Example 3: Differentiation of MSCs from NCCs Expanded Under Xeno-Free Conditions

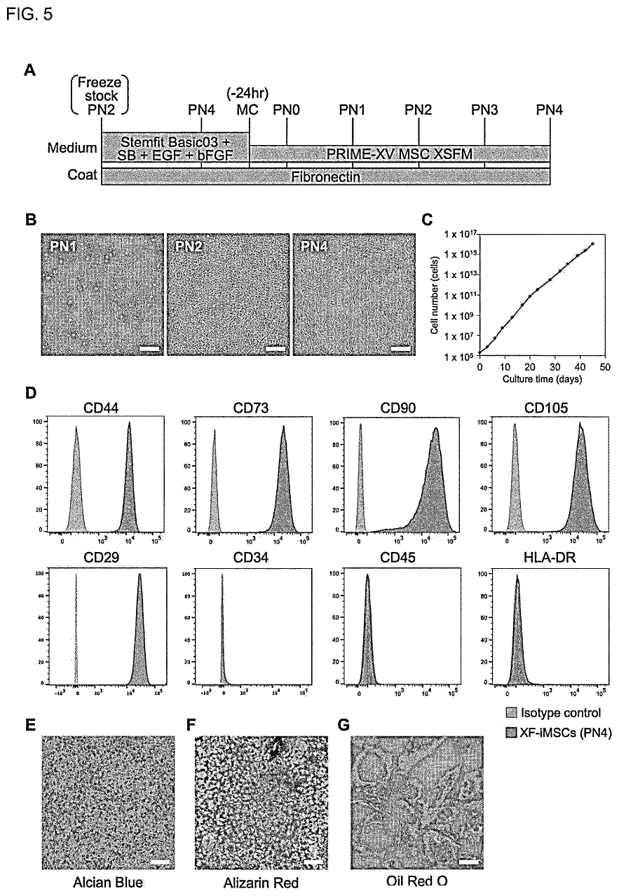

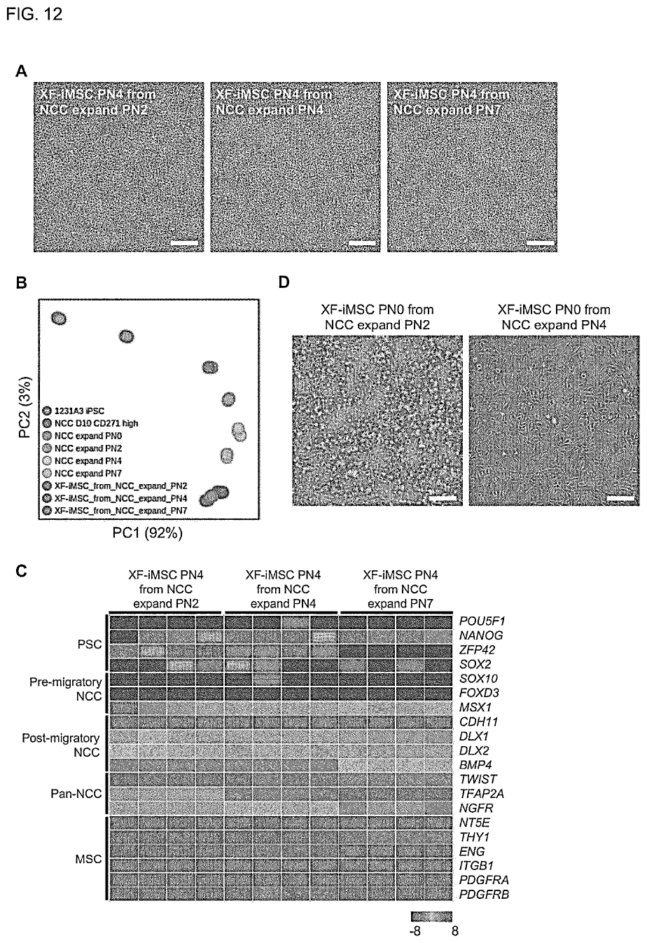

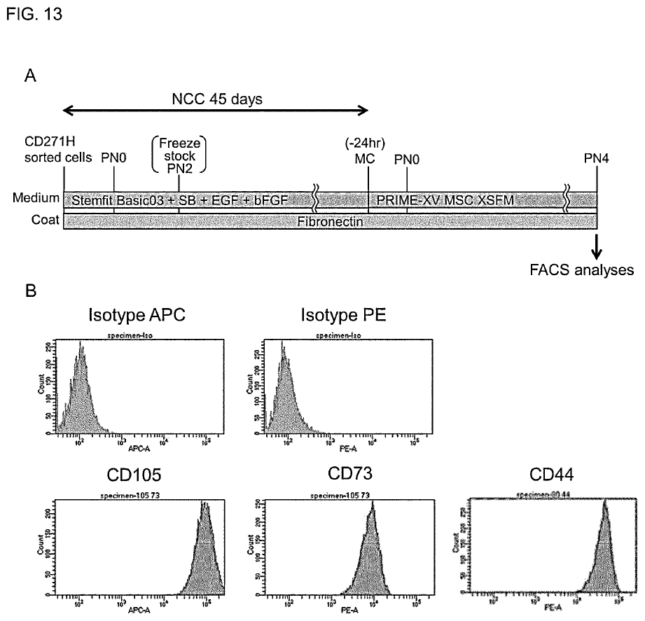

When NCCs with serum-containing medium or xeno-free MSC medium (PRIME-XV (registered trademark) MSC Expansion XSFM) were cultured (FIG. 5A), NCCs at passage 1 (=4 days) and passage 4 (=14 days) proliferated exponentially (FIG. 5B, C). In addition, NCCs at passage 7 (=30 days) also proliferated exponentially (data not shown). They also showed a morphology (FIG. 12A) and gene expression profile consistent with MSCs (FIG. 12B, C). However, compared to cells derived from NCCs at passage 4, a significant number of cells died within a few days when induced from NCCs at passage 2 (FIG. 12D). Therefore, for further characterization, cells from NCCs at passage 4 were used. The cells were positive for MSC markers such as CD44, CD73, CD90, and CD105, and negative for CD45 and HLA-DR at 4 passages (=14 days) (FIG. 5D). MSC surface markers were checked, and it was confirmed that cells derived from NCCs at passage 4 (=14 days) were positive for CD44, CD73, CD90, CD105, and CD29 and negative for CD34, CD45 and HLA-DR at passage 4 (FIG. 5D). MSCs induced at passage 4 (=14 days) (xeno-free-induced MSCs derived from NCCs (XF-iMSCs, hereafter)) could differentiate into cartilage, bone, and adipose under chondrogenic, osteogenic, and adipogenic induction conditions, respectively (FIG. 5E-G). Furthermore, when iMSCs were induced in the same manner after culturing in NCC expansion culture medium for 45 days, it was confirmed that the induced cells expressed MSC surface antigens CD105, CD73 and CD44 (FIG. 13). Therefore, it was shown that NCCs having the ability to differentiate into MSCs can be maintained for a long period of time (at least 45 days or more), by the culture method of the present invention.

### Example 4: Comparison of XF-iMSCs and Tissue-Derived MSCs

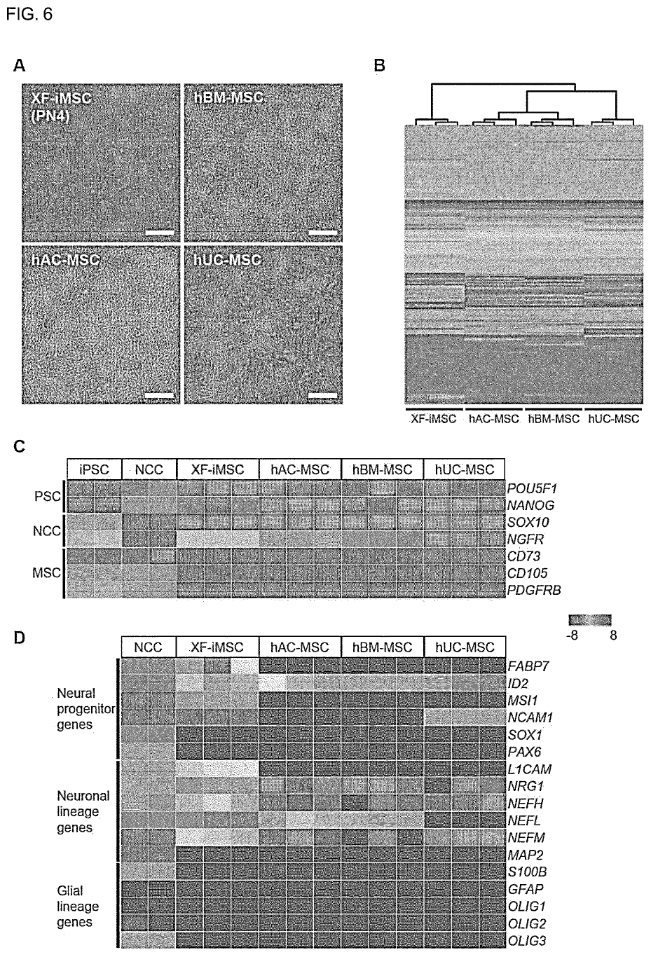

MSCs isolated from different tissues or prepared by different methods have different characteristics. Therefore, XF-iMSCs were compared with various types of MSCs. First, a transcriptome analysis of XF-iMSCs and tissue-derived primary human MSCs (bone marrow-derived MSCs (hBM-MSCs), adipose-derived MSCs (hAC-MSCs), and umbilical cord-derived MSCs (hUC-MSCs)) was performed (FIG. 6A). As a result, it was revealed that although differences in morphology and gene expression were observed between these cells (only XF-iMSCs expressed neural progenitor genes and neural lineage genes), there were substantial similarities in the MSC marker expression and global gene expression profiles (FIGS. 6B-D and 14). These data suggest that the expansion of NCCs restricts their fate to mesenchymal lineages.

### Example 5: Contribution of XF-iMSCs to Skull Bone Regeneration

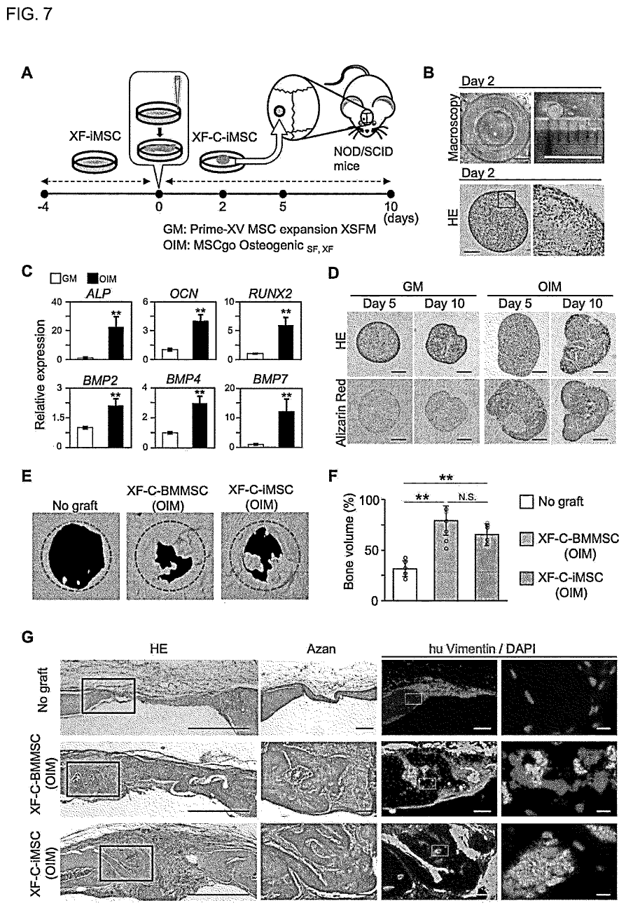

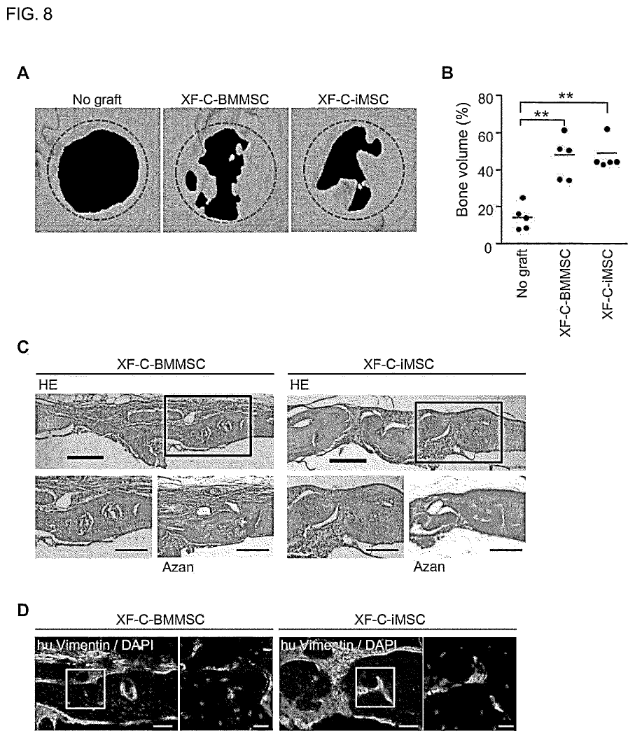

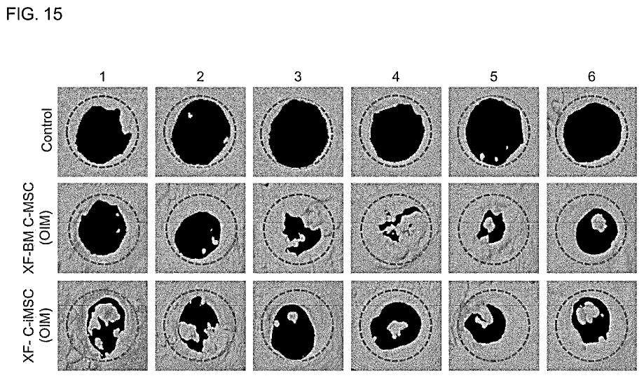

To investigate the function of XF-iMSCs, their in vitro differentiation potential and in vivo regeneration ability for bone was examined. XF-iMSCs were cultured in a 48-well tissue culture plate with xeno-free MSC medium for 4 days, manually detached from wells as sheets, and cultured for 2 days in xeno-free MSC medium (GM) or osteocyte induction medium (OIM) to allow for the formation of clumps (XF-Clump-iMSCs; XF-C-iMSCs, hereafter) (FIG. 7A and B) (Int J Mol Sci 20 (2019)). On day 2, the clumps were 1 mm in diameter and contained a rich extracellular matrix (FIG. 7B). After being cultured in xeno-free osteogenic medium for three more days, XF-C-iMSCs expressed osteogenic markers (ALP, OCN, RUNX2, BMP2, BMP4, and BMP7) (FIG. 7C). In vitro osteogenic differentiation was confirmed by alizarin red staining on day 5 (osteogenic induction for three days) and day 10 (osteogenic induction for 8 days) (OIM in FIG. 7D). To assess the bone regeneration ability, XF-C-iMSCs (OIM) at 5 days were transplanted into a 1.6-mm hole of the skull bone of immunodeficient non-obese diabetic/severe combined immunodeficiency (NOD/SCID) mice. In this Example, cell aggregations of hBM-MSCs (XF-C-BMMSCs (OIM)) maintained in xeno-free MSC medium and induced to osteogenesis were used as a positive control. Four weeks after the transplantation, mineralized areas were recovered by the XF-C-iMSCs (OIM) transplantation, similar to XF-C-BMMSCs (FIG. 7E, F, FIG. 15). Although histological and immunohistochemical analyses revealed the contribution of iMSCs and hBM-MSCs in regenerated bone regions, the majority of regenerated bone was negative for anti-human-specific vimentin in both cases (FIG. 7G). Similar results were observed when XF-C-iMSCs cultured with MSC medium were transplanted (FIG. 8). These results suggested that, although XF-iMSCs could differentiate into bone in vivo, they exerted a paracrine effect on skull regeneration.

### Example 6: Enhanced Skeletal Muscle Regeneration by XF-iMSC Transplantation

To assess the impact of iMSCs on the regeneration of other tissues, XF-iMSCs were transplanted into the tibialis anterior (TA) muscle crush model of immunodeficient NOD/SCID/IL2Rgamma null (NSG) mice (FIG. 9A). After 24 h, XF-iMSCs and human dermal fibroblasts (HDFs) were transplanted into TA-injured mice. Three days after injury (2 days after transplantation), muscle fibers were degenerated at the injury site, and we observed no major histological differences between the control groups (medium injection and HDF transplantation groups) and the XF-iMSC transplantation group (FIG. 9B). After 2 weeks, there were also no differences among the groups in terms of histology and the average cross-sectional area of muscle fibers (FIG. 9B and C). At 5 weeks, however, the average cross-sectional area of muscle fibers was significantly larger in the XF-iMSC transplantation group than those in control groups and was comparable to that of intact muscle fibers, although nuclei were found in the center of each muscle fiber. These results suggest that XF-iMSC transplantation encourages accelerated skeletal muscle regeneration.

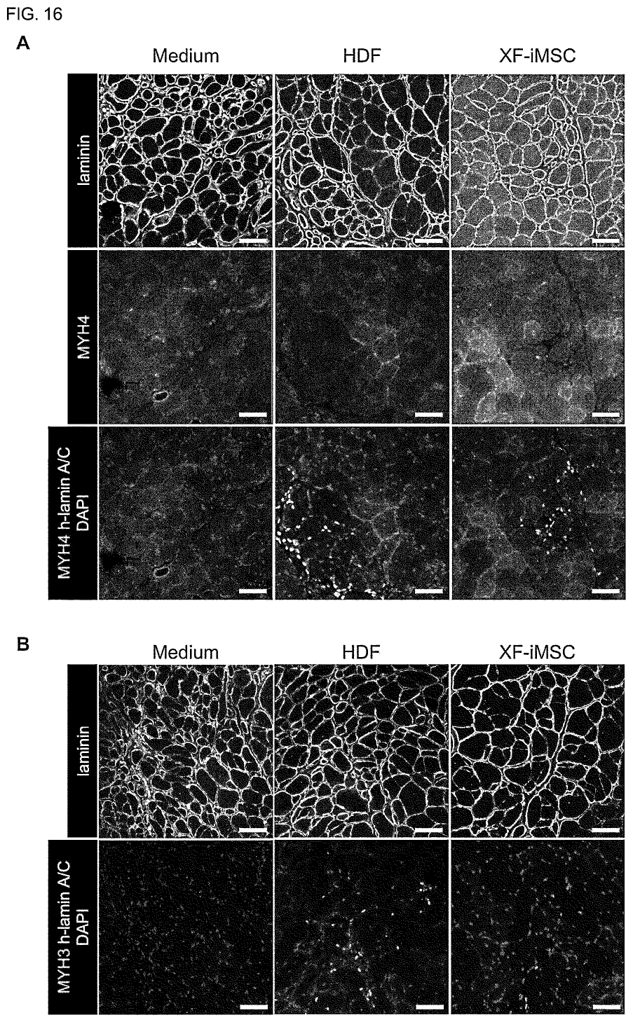

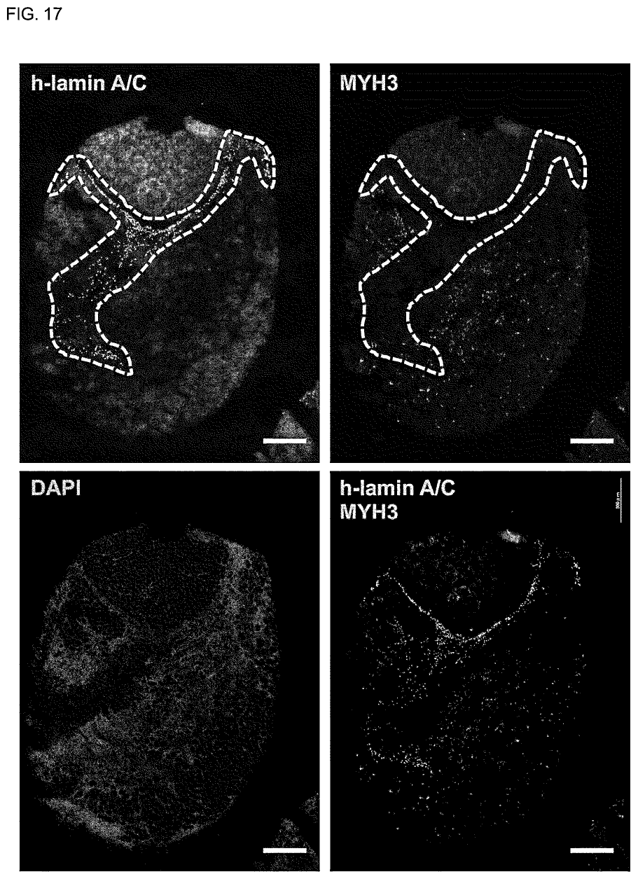

For the molecular characterization of the regeneration processes, immunohistochemical analyses were performed with anti-MYH4 (a mature muscle fiber marker) and anti-MYH3 (an embryonic and fetal muscle fiber marker, also expressed in regenerating muscle fibers) antibodies as well as anti-laminin antibody (basement membrane marker). At 5 weeks, muscle fibers with large diameters were stained with MYH4, which is consistent with MYH4 being a marker for mature skeletal muscle (FIG. 16A). No staining was detected with anti-MYH3 antibody, which is consistent with MYH3 being a marker expressed in the early phase of muscle regeneration (FIG. 16B). At 2 weeks, although anti-laminin staining revealed that the cross-sectional area of skeletal muscle cells in the XF-iMSC-transplanted group was smaller than that in intact skeletal muscle, the number of MYH4-positive cells was increased compared with the control groups (FIG. 9D and E). The nuclei of MYH4-positive cells did not co-stain with human-specific lamin A/C (h-lamin A/C), which marks the nucleus of transplanted human cells, suggesting that transplanted XF-iMSCs did not differentiate into skeletal muscle cells. During the early stages, on day 3, MYH3-positive cells were increased in the XF-iMSC-transplanted group compared with those in the control groups (FIG. 9F and G). After 1 week, the number of MYH3-positive cells was higher in the XF-iMSC transplantation group than in the control group. In contrast, the number of MYH3-positive cells was lower in the XF-iMSC transplantation group than in the control group after 2 weeks. These data suggest that XF-iMSC transplantation promotes early recovery from muscle damage. Although the nuclei of MYH3-positive cells was never co-stained with h-lamin A/C, the majority of MYH3-positive cells were tended to localize near the h-lamin A/C-positive cells at day 3 (FIG. 17). These data suggest that the contribution of XF-iMSCs to muscle regeneration occurs via paracrine factors.

### Example 7: Accelerated Myotube Differentiation by XF-iMSC-Conditioned Medium In Vitro

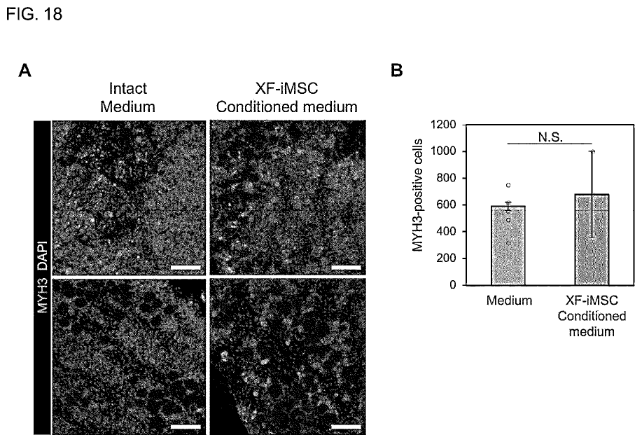

To check whether soluble factors secreted by XF-iMSCs regulate the early activation of MYH3 in vivo, XF-iMSC-conditioned medium was injected into injured TA muscle. Although a subtle increase was observed in the number of MYH3-positive cells (FIG. 18), there was no significant difference between intact and conditioned media.

### INDUSTRIAL APPLICABILITY

According to the present invention it is possible to produce neural crest cells specialized for differentiation into a mesenchymal lineage, the neural crest cells being useful as starting cells for mesenchymal stem cells or mesenchymal cells. The mesenchymal stem cells or mesenchymal cells obtained by the invention are useful not only for direct regeneration of damaged tissue but also for regenerative medicine, since indirect effects are exhibited by factors secreted by those cells.

The present application claims priority based on Japanese Patent Application No. 2021-198151 (filed in Japan on Dec. 6, 2021), the entirety of the disclosure of which is incorporated herein by reference.

1. A method for producing neural crest cells specialized for differentiation into a mesenchymal lineage from pluripotent stem cells, comprising:
1) a step of culturing pluripotent stem cells in culture medium containing an ALK inhibitor and a GSK-3β inhibitor under xeno-free and feeder-free conditions to obtain neural crest cells, and
2) a step of culturing the neural crest cells in culture medium containing an ALK inhibitor, EGF and FGF but containing substantially no GSK-3β inhibitor, under xeno-free and feeder-free conditions.

2. The method according to claim 1, wherein the culturing period in step 2) is 7 to 45 days.

3. The method according to claim 1, wherein at least one ALK inhibitor used in step 2) is SB431542.

4. The method according to claim 1, wherein the culturing in step 1) and/or step 2) is adhesion culturing.

5. The method according to claim 1, wherein the pluripotent stem cells are induced pluripotent stem cells or embryonic stem cells.

6. The method according to claim 1, wherein the pluripotent stem cells are derived from a human.

7. Neural crest cells obtained by the method according to claim 1.

8. Neural crest cells having the following features (A) to (C).
(A) the cells are derived from pluripotent stem cells.
(B) the cells express one or more genes selected from among TWIST, DLX1 and CDH11.
(C) the cells do not express PAX3 and/or SOX10.

9. Neural crest cells according to claim 8, further having the following features (D) and/or (E).
(D) the cells do not have differentiation potency to nervous system cells.
(E) the cells do not have differentiation potency to melanocytes.

10. A method for producing mesenchymal stem cells, comprising a step of culturing neural crest cells according to claim 8 in mesenchymal stem cell differentiation-inducing medium.

11. Mesenchymal stem cells obtained by the method according to claim 10.

12. A method for producing mesenchymal cells, comprising a step of culturing mesenchymal stem cells according to claim 11 in mesenchymal cell differentiation-inducing medium.

13. Mesenchymal cells obtained by the method according to claim 12.

14. A method for treating tissue damage or disease of a mammal, comprising administrating or transplanting an effective amount of mesenchymal stem cells according to claim 11 into the mammal.


SEQUENCE LISTING (40 sequences)

SEQ ID NO: 1 - DNA, 20 bp, synthetic construct
  misc_feature 1..20: note = PCR Primer(NGFR-sense)
  source 1..20: mol_type = other DNA
ccgttggattacacggtcca

SEQ ID NO: 2 - DNA, 20 bp, synthetic construct
  misc_feature 1..20: note = PCR Primer(NGFR-antisense)
  source 1..20: mol_type = other DNA
gacagggatgaggttgtcgg

SEQ ID NO: 3 - DNA, 22 bp, synthetic construct
  misc_feature 1..22: note = PCR Primer(SOX10-sense)
  source 1..22: mol_type = other DNA
gagctggaccgcacaccttggg

SEQ ID NO: 4 - DNA, 22 bp, synthetic construct
  misc_feature 1..22: note = PCR Primer(SOX10-antisense)
  source 1..22: mol_type = other DNA
aacgcccacctcctcggacctc

SEQ ID NO: 5 - DNA, 20 bp, synthetic construct
  misc_feature 1..20: note = PCR Primer(TFAP2A-sense)
  source 1..20: mol_type = other DNA
agggcctcggtgagatagtt

SEQ ID NO: 6 - DNA, 20 bp, synthetic construct
  misc_feature 1..20: note = PCR Primer(TFAP2A-antisense)
  source 1..20: mol_type = other DNA
aagagttcaccgacctgctg

SEQ ID NO: 7 - DNA, 19 bp, synthetic construct
  misc_feature 1..19: note = PCR Primer(RHOB-sense)
  source 1..19: mol_type = other DNA
atccccgagaagtgggtcc

SEQ ID NO: 8 - DNA, 21 bp, synthetic construct
  misc_feature 1..21: note = PCR Primer(RHOB-antisense)
  source 1..21: mol_type = other DNA
cgaggtagtcgtaggcttgga

SEQ ID NO: 9 - DNA, 20 bp, synthetic construct
  misc_feature 1..20: note = PCR Primer(PAX3-sense)
  source 1..20: mol_type = other DNA
cggcatcctgagcgagcgag

SEQ ID NO: 10 - DNA, 20 bp, synthetic construct
  misc_feature 1..20: note = PCR Primer(PAX3-antisense)
  source 1..20: mol_type = other DNA
actcgggcctcggtgagctt

SEQ ID NO: 11 - DNA, 22 bp, synthetic construct
  misc_feature 1..22: note = PCR Primer(SNAI2-sense)
  source 1..22: mol_type = other DNA
tgtgacaaggaatatgtgagcc

SEQ ID NO: 12 - DNA, 21 bp, synthetic construct
  misc_feature 1..21: note = PCR Primer(SNAI2-antisense)
  source 1..21: mol_type = other DNA
tgagccctcagatttgacctg

SEQ ID NO: 13 - DNA, 20 bp, synthetic construct
  misc_feature 1..20: note = PCR Primer(CDH6-sense)
  source 1..20: mol_type = other DNA
ctgcgacggatgcagatgat

SEQ ID NO: 14 - DNA, 23 bp, synthetic construct
  misc_feature 1..23: note = PCR Primer(CDH6-antisense)
  source 1..23: mol_type = other DNA
ccctgttttctcgatccatgttg

SEQ ID NO: 15 - DNA, 21 bp, synthetic construct
  misc_feature 1..21: note = PCR Primer(PAX6-sense)
  source 1..21: mol_type = other DNA
ctggctagcgaaaagcaacag

SEQ ID NO: 16 - DNA, 25 bp, synthetic construct
  misc_feature 1..25: note = PCR Primer(PAX6-antisense)
  source 1..25: mol_type = other DNA
cccgttcaacatccttagtttatca

SEQ ID NO: 17 - DNA, 20 bp, synthetic construct
  misc_feature 1..20: note = PCR Primer(TWIST-sense)
  source 1..20: mol_type = other DNA
gtccgcagtcttacgaggag

SEQ ID NO: 18 - DNA, 22 bp, synthetic construct
  misc_feature 1..22: note = PCR Primer(TWIST-antisense)
  source 1..22: mol_type = other DNA
gcttgagggtctgaatcttgct

SEQ ID NO: 19 - DNA, 21 bp, synthetic construct
  misc_feature 1..21: note = PCR Primer(DLX1-sense)
  source 1..21: mol_type = other DNA
tgccagaaagtctcaacagcc

SEQ ID NO: 20 - DNA, 21 bp, synthetic construct
  misc_feature 1..21: note = PCR Primer(DLX1-antisense)
  source 1..21: mol_type = other DNA
cgagtgtaaacagtgcatgga

SEQ ID NO: 21 - DNA, 21 bp, synthetic construct
  misc_feature 1..21: note = PCR Primer(CDH11-sense)
  source 1..21: mol_type = other DNA
agagagcccagtacacgttga

SEQ ID NO: 22 - DNA, 21 bp, synthetic construct
  misc_feature 1..21: note = PCR Primer(CDH11-antisense)
  source 1..21: mol_type = other DNA
ttggcatgataggtctcgtgc

SEQ ID NO: 23 - DNA, 24 bp, synthetic construct
  misc_feature 1..24: note = PCR Primer(POU5F1-sense)
  source 1..24: mol_type = other DNA
gacagggggaggggaggagctagg

SEQ ID NO: 24 - DNA, 26 bp, synthetic construct
  misc_feature 1..26: note = PCR Primer(POU5F1-antisense)
  source 1..26: mol_type = other DNA
cttccctccaaccagttgccccaaac

SEQ ID NO: 25 - DNA, 18 bp, synthetic construct
  misc_feature 1..18: note = PCR Primer(ALP-sense)
  source 1..18: mol_type = other DNA
gcggtgaacgagagaatg

SEQ ID NO: 26 - DNA, 19 bp, synthetic construct
  misc_feature 1..19: note = PCR Primer(ALP-antisense)
  source 1..19: mol_type = other DNA
cgtagttctgctcgtgcac

SEQ ID NO: 27 - DNA, 18 bp, synthetic construct
  misc_feature 1..18: note = PCR Primer(OCN-sense)
  source 1..18: mol_type = other DNA
gtgacgagttggctgacc

SEQ ID NO: 28 - DNA, 19 bp, synthetic construct
  misc_feature 1..19: note = PCR Primer(OCN-antisense)
  source 1..19: mol_type = other DNA
tggagaggagcagaactgg

SEQ ID NO: 29 - DNA, 20 bp, synthetic construct
  misc_feature 1..20: note = PCR Primer(RUNX2-sense)
  source 1..20: mol_type = other DNA
actaccagccaccgagacca

SEQ ID NO: 30 - DNA, 24 bp, synthetic construct
  misc_feature 1..24: note = PCR Primer(RUNX2-antisense)
  source 1..24: mol_type = other DNA
actgcttgcagccttaaatgactc

SEQ ID NO: 31 - DNA, 18 bp, synthetic construct
  misc_feature 1..18: note = PCR Primer(BMP2-sense)
  source 1..18: mol_type = other DNA
ctgtatcgcaggcactca

SEQ ID NO: 32 - DNA, 19 bp, synthetic construct
  misc_feature 1..19: note = PCR Primer(BMP2-antisense)
  source 1..19: mol_type = other DNA
ctccgtggggatagaactt

SEQ ID NO: 33 - DNA, 25 bp, synthetic construct
  misc_feature 1..25: note = PCR Primer(BMP4-sense)
  source 1..25: mol_type = other DNA
cacagcactggtcttgagtatcctg

SEQ ID NO: 34 - DNA, 25 bp, synthetic construct
  misc_feature 1..25: note = PCR Primer(BMP4-antisense)
  source 1..25: mol_type = other DNA
ctcagggatgctgctgaggttaaag

SEQ ID NO: 35 - DNA, 20 bp, synthetic construct
  misc_feature 1..20: note = PCR Primer(BMP7-sense)
  source 1..20: mol_type = other DNA
ccacctgtaatcccagcact

SEQ ID NO: 36 - DNA, 20 bp, synthetic construct
  misc_feature 1..20: note = PCR Primer(BMP7-antisense)
  source 1..20: mol_type = other DNA
gacaaagaccagagggtcca

SEQ ID NO: 37 - DNA, 20 bp, synthetic construct
  misc_feature 1..20: note = PCR Primer(18S-sense)
  source 1..20: mol_type = other DNA
gtaacccgttgaaccccatt

SEQ ID NO: 38 - DNA, 20 bp, synthetic construct
  misc_feature 1..20: note = PCR Primer(18S-antisense)
  source 1..20: mol_type = other DNA
ccatccaatcggtagtagcg

SEQ ID NO: 39 - DNA, 22 bp, synthetic construct
  misc_feature 1..22: note = PCR Primer(ACTB-sense)
  source 1..22: mol_type = other DNA
aggtctttgcggatgtccacgt

SEQ ID NO: 40 - DNA, 22 bp, synthetic construct
  misc_feature 1..22: note = PCR Primer(ACTB-antisense)
  source 1..22: mol_type = other DNA
caccattggcaatgagcggttc